(19) United States
(12) Patent Application Publication
(10) Pub. No.: US 2024/0042016 A1
(43) Pub. Date: Feb. 8, 2024

(54) NANOEMULSION VACCINE COMPOSITIONS AND METHODS FOR SUPPRESSING REACTIVITY TO MULTIPLE FOOD ALLERGENS

(71) Applicant: The Regents of the University of Michigan, Ann Arbor, MI (US)

(72) Inventors: James R. Baker, JR., Ann Arbor, MI (US); Jessica O'Konek, Ann Arbor, MI (US)

(21) Appl. No.: 18/267,640

(22) PCT Filed: Dec. 29, 2021
(86) PCT No.: PCT/US21/65576

§ 371 Date: Jun. 15, 2023

Related U.S. Application Data
(60) Provisional application No. 63/132,199, filed on Dec. 30, 2020.

(51) Int. Cl. A61K 39/35; A61K 9/107; A61P 37/08

(52) U.S. Cl. CPC A61K 39/35; A61K 9/1075; A61P 37/08; A61K 2039/577

(57) ABSTRACT

The disclosure is directed to compositions and methods for inhibiting an allergic reaction to two or more food allergens. The compositions comprise a nanoemulsion and at least one of the two or more food allergens.

### FIELD

This disclosure relates to compositions and methods for suppressing allergic responses and inducing bystander suppression of reactivity to multiple food allergens.

### BACKGROUND

Food allergy is an emerging epidemic that now affects up to 15 million people in the United States, including 8% of children. The economic burden of food allergy in the U.S. alone exceeds $24.8 billion (1). Allergen-specific immunotherapy for food allergy has been the primary approach to suppress allergic reactivity, and involves the progressive administration of increasing amounts of a specific allergen by one of several routes. This approach, however, does not provide long-term protection following cessation of therapy and requires prolonged treatment protocols that are burdening to patients and their families. For example, subcutaneous immunotherapy to food allergens showed promise for protection against IgE-mediated food allergies; however, significant adverse reactions limited successful implementation (2-4). Sublingual, oral (OIT), and epicutaneous immunotherapy have demonstrated efficacy in animal models and human trials, but these approaches desensitize only a portion of patients and the protection achieved is rapidly lost after cessation of the therapy (5-11).

Thirty to forty percent of patients with food allergies are sensitized to multiple foods (37, 38). While allergen-specific immunotherapy has the potential to relieve the burden of fear of reactivity to specific foods, allergen-specific immunotherapy is more difficult for polysensitized individuals. Approved allergen-specific immunotherapy for food allergy involves a single food, and regulatory issues may preclude the development of therapies targeted against multiple foods. While some studies have demonstrated the ability to desensitize patients with OIT for up to 5 foods simultaneously (multi-OIT) (39, 40), the amount of each food required to be consumed daily is a burden for some children, as the food required for multi-OIT can be a significant proportion of the daily caloric intake for a child.

The primary immunologic mechanism of allergic hypersensitivity is the induction of Th2-polarized cellular immune responses leading to the production of allergen-specific IgE antibodies critical for mast cell activation. Th2 cytokines also are critical mediators of local allergic inflammation, including IL-4 and IL-13-dependent mucus production and IL-5-mediated eosinophil recruitment (12). Oral or subcutaneous allergen immunotherapy (AIT) appears to achieve desensitization to the allergen by temporarily reducing Th2-biased immunity and allergen-specific IgE. While AIT/OIT has been proven clinically useful for treating food allergy, it has not induced a long-term redirection of allergen-specific immunity away from a Th2 phenotype (13). Thus, interest has been directed toward new strategies that are able to permanently suppress Th2 cellular immune responses or redirect these cellular Th2 responses towards a Th1 phenotype (14, 15).

Thus, there remains a need for compositions and methods that effectively treat food allergies, particularly allergies to multiple foods.

### BRIEF SUMMARY

The disclosure provides a method of inhibiting an allergic reaction to two or more food allergens in a subject, which comprises administering to the subject a composition comprising a nanoemulsion and at least one of the two or more food allergens. In some embodiments, the composition comprises only one of the two or more food allergens.

### BRIEF DESCRIPTION OF THE DRAWING(S)

FIG. 1 is a series of images illustrating the gating strategy for analysis of ILC2 cells from small intestine (SI). As described in the Examples, SI cells were stained with lineage antibody cocktail, cell surface markers for ILC2, and transcription factor GATA3. Briefly, live CD45+ cells were gated (gate B). Further, lineage negative CD45+ cells (gate C) were gated for CD127+ and CD90+ cells (gate D). This total ILC population was gated for cell surface marker of ILC2 KLRG-1 μgainst the transcription factor GATA3 to identify a double positive ILC2 cell population (gate E).

FIG. 2A is a schematic diagram of the sensitization-immunotherapy-challenge experiments described in Example 1. Mice were sensitized with ova and peanut-alum and treated i.n. with 3 administrations of PBS (sensitized control) or ova and peanut-NE (OVA+PN−NE). Mice were challenged orally with ova and peanut. FIGS. 2B-2D are graphs showing symptoms of anaphylaxis (FIG. 2B), diarrhea (FIG. 2C) and temperature change (FIG. 2D) in treated mice. FIG. 2E is a graph of hemoconcentration as determined by hematocrit. FIG. 2F is a graph showing levels of MCPT-1 in the serum 60 minutes after challenge as determined by ELISA. Statistically significant differences (p<0.05) are indicated by *.

FIGS. 3A and 3B are a series of graphs showing that immunization of polysensitized mice with a nanoemulsion and one allergen provides protection against reactivity to another allergen. In FIG. 3A, mice were challenged orally with ova and temperature change and symptoms of anaphylaxis were monitored. Hemoconcentration was determined by hematocrit. Levels of MCPT-1 in the serum 60 minutes after challenge were determined by ELISA. In FIG. 3B, mice were challenged orally with peanut and temperature change and symptoms of anaphylaxis were monitored. Hemoconcentration was determined by hematocrit. Levels of MCPT-1 in the serum 60 minutes after challenge were determined by ELISA. Statistically significant differences (p<0.05) are indicated by *.

FIGS. 4A-4F are graphs illustrating that intranasal administration of a nanoemulsion compositions without allergen does not suppress the allergic response. Mice were sensitized with ova and peanut-alum and treated i.n. with 3 administrations of (A-C) PBS (sensitized control), ova-NE (ova-NE) or NE only (no antigen) or (D-F) PBS (sensitized control) or hepatitis B surface antigen-NE (HBsAg-NE). Mice were challenged orally with ova and temperature change and symptoms of anaphylaxis were monitored. Serum MCPT-1 levels were determined by ELISA. Statistically significant differences (p<0.05) are indicated by *.

FIG. 5A is a graph showing serum ova-specific IgE expression in treated mice as measured by ELISA. FIG. 5B includes graphs showing ova-specific cytokine secretion (IL-4, IL-13, and IFN-γ) determined in cultures of mLN lymphocytes. FIG. 5C includes graphs showing relative gene expression of 1125, 1133 and Tslp as compared to GAPDH in mRNA extracted from duodenum samples. FIG. 5D is a graph showing the total number of ILC2 (Lin-CD45+CD127+CD90.2+KLRG-1+538 GATA3+) from SI. Statistically significant differences (p<0.05) are indicated by *.

FIG. 6A is a schematic diagram outlining sensitization of mice with ova and peanut-alum, followed by i.n. treatment with 3 administrations of PBS (sensitized control) or peanut-NE (PN-NE). Mice were challenged orally with ova and IFN-γ was depleted during the challenge phase. FIGS. 6B-6D are graphs showing temperature change (FIG. 6B), symptoms of anaphylaxis (FIG. 6C), and diarrhea (FIG. 6D) in treated mice. FIG. 6E is a graph of hemoconcentration as determined by hematocrit. FIG. 6F is a graph showing levels of MCPT-1 in the serum 60 minutes after challenge were determined by ELISA. Statistically significant differences (p<0.05) are indicated by *.

FIG. 7 provides graphs that indicate IFN-γ is required for suppression of alarmins by NE allergy vaccines. Mice were sensitized with OVA and peanut-alum and treated i.n. with 3 administrations of PBS (sensitized control) or peanut-NE (PN-NE). Mice were challenged orally with OVA and IFN-γ was depleted during the challenge phase. Duodenum samples were homogenized and mRNA was extracted to determine relative gene expression compared to GAPDH. Statistically significant differences (p<0.05) are indicated by *.

FIG. 8 is a schematic showing an experimental schedule using a single major peanut allergen vaccine. All mice were sensitized by 6 oral gavages of peanut extract and cholera toxin. Mice received 3 doses of intranasal (i.n.) vaccine containing the indicated treatments. Mice were subjected to an oral peanut challenge beginning 2 weeks after the last intranasal treatment.

FIG. 9 shows graphs documenting the suppression of reactivity to oral peanut challenge by a single peanut allergen (ara h protein) vaccines. Following oral peanut challenge, mice were monitored for core body temperature change (FIG. 9A) and clinical symptoms (FIG. 9B). The scoring system for anaphylaxis was as follows: 0—no reaction, 1—itching; 2—edema around eyes or snout, reduced activity, hunched/scruffy, and/or diarrhea; 3—wheezing and/or labored respiration; 4—no activity or response to prodding; 5—death.

### DETAILED DESCRIPTION

The present disclosure is predicated, at least in part, on the discovery that anaphylactic reactions to food allergens can be suppressed using allergen-specific immunotherapy without having to eliminate allergen-specific IgE. In addition, modulation of Th2 immunity towards a single antigen can induce bystander effects that suppress reactivity to other allergens through the induction of IFN-γ and suppression of alarmins and type 2 innate lymphoid cell (ILC2) populations in the intestine. The disclosure further provides that a composition (e.g. a vaccine) comprising a nanoemulsion and an individual allergen (e.g., a single major peanut allergen, such as ara h 2) from a food allergen comprising a plurality of individual allergen components (e.g., peanut) suppresses reactivity to challenge with whole allergen (e.g., a whole extract containing the single allergen plus the remainder of individual allergen components, such as whole peanut extract). Accordingly, disclosed herein are vaccine compositions comprising a nanoemulsion and a single food allergen that provide global suppression of allergic responses (e.g., to multiple allergens). Also disclosed herein are methods for modulating (e.g., inhibiting) allergic reaction (e.g., Th2 reaction) to a whole food allergen (e.g., peanut) comprising a plurality of individual allergen components utilizing a nanoemulsion and at least one of the plurality of individual allergen components. Compositions and methods of the disclosure provide an allergen-non-specific protection and/or protection from a whole food allergen comprising a plurality of individual allergen components that may also be induced by or augmented by other therapies that increase IFN-γ, decrease expression of alarmins in the gut mucosa, and/or decrease type 2 innate lymphoid cells (ILC2) in the absence of having to eliminate allergen-specific IgE.

### Definitions

To facilitate an understanding of the present technology, a number of terms and phrases are defined below. Additional definitions are set forth throughout the detailed description.

The terms “disease” and “pathologic condition” are used interchangeably herein to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, hyper-immune responses, hyper-sensitivity, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., allergens, malnutrition, industrial hazards, and/or climate), may be responsive to an inherent defect of the organism (e.g., genetic anomalies) or to combinations of these and other factors.

The term “allergy,” as used herein, refers to a chronic condition involving an abnormal or pathological immune reaction to a substance (i.e., an “allergen”) that is ordinarily harmless in average/healthy individuals. An “allergen” refers to any substance (e.g., an antigen) that induces an allergic reaction in a subject. Examples of allergens include, but are not limited to, food products (e.g., milk, egg, soy, tree nut, peanut, wheat, or fish proteins), aeroallergens (e.g., dust mite, mold, spores, plant pollens such as tree, weed, and grass pollens), animal products (e.g., cat or dog hair), drugs (e.g., penicillin), insect venom, and latex.

The term “food allergy,” as used herein, refers to a pathological reaction of the immune system triggered by the ingestion of a food protein antigen. Exposure to very small amounts of allergenic foods can trigger clinical symptoms such as gastrointestinal disorders, urticaria, and airway inflammation, ranging in severity from mild to life-threatening. Food allergy is distinct from food intolerance in that intolerance does not arise from immune system dysregulation; for example, lactose intolerance arises from non-immune factors, such as lactose malabsorption and lactase deficiency (Yu et al., Nature Reviews Immunology, 16: 751-765 (2016))

The terms “host” or “subject,” as used herein, refer to an individual to be treated by (e.g., administered) the compositions and methods of the present disclosure. Subjects include, but are not limited to, mammals (e.g., murines, simians, equines, bovines, porcines, canines, felines, and the like), and preferably humans. In the context of the present disclosure, the term “subject” generally refers to an individual who will be administered or who has been administered one or more compositions described herein (e.g., a composition comprising a nanoemulsion and one or more food allergens).

The term “emulsion,” as used herein, includes classic oil-in-water or water-in-oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in some embodiments, nanoemulsions may comprise an oil phase having droplets with a mean particle size of approximately 0.1 to 5 microns (e.g., about 150, 200, 250, 300, 350, 400, 450, 500 nm or larger in diameter), although smaller and larger particle sizes are contemplated. The terms “emulsion” and “nanoemulsion” may be used interchangeably herein to refer to the nanoemulsions of the present disclosure.

The terms “surface active agent” and “surfactant,” are used interchangeably herein and refer to amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic, and polymeric surfactants.

A used herein, the term “immune response” refers to a response by the immune system of a subject. Immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), natural killer (NK) cell activation, and/or B cell activation (e.g., antibody generation and/or secretion). Additional examples of immune responses include binding of an immunogen (e.g., antigen (e.g., immunogenic polypeptide)) to a major histocompatibility complex (MHC) molecule and inducing a cytotoxic T lymphocyte (“CTL”) response, inducing a B cell response (e.g., antibody production), T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells)), and increased processing and presentation of antigen by antigen presenting cells. An immune response may be directed against immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids)). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom, or condition of the disease) upon exposure to a substance or organism (e.g., antigen, allergen, or pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the terms “immunogen” and “antigen” refer to an agent (e.g., a protein, an allergen, or a microorganism and/or portion or component thereof (e.g., a protein antigen (e.g., gp120 or rPA))) that is capable of eliciting an immune response in a subject. In preferred embodiments, immunogens elicit immunity against the immunogen (e.g., allergen) when administered in combination with a nanoemulsion of the present disclosure.

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, the nanoemulsion formulations described herein, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi), and threonyl-muramyl dipeptide (t-MDP; Ribi); OM-174 (a glucosamine disaccharide related to lipid A; OM Pharma S A, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present disclosure (e.g., comprising a nanoemulsion and one or more food allergens) are administered with one or more adjuvants (e.g., to skew the immune response towards a Th1 and/or Th2 type response).

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route. Accordingly, a “therapeutically effective amount” (e.g., of a composition for inducing an immune response), refers to the dosage level or amount of a composition required (e.g., when administered to a subject) to stimulate, generate and/or elicit a therapeutic benefit in a subject. An therapeutically effective amount can be administered in one or more administrations (e.g., via the same or different route), applications, or dosages, and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

### Food Allergies

Allergic diseases are associated with aberrant immune responses. For example, epithelial cells play an important role in orchestrating the allergic response, such as airway inflammation, through the release of multiple cytokines, including stem cell factor and several chemokines that attract eosinophils. The most common food allergies, including milk, egg, wheat, soy, peanut, tree nuts, shellfish, and fish allergies are IgE-mediated. IgE-mediated food allergies are associated with a risk of severe or fatal reactions, and accordingly, it is the most fully characterized type of food allergy.

In food allergen-sensitized individuals, subsequent exposure to the food allergen triggers IgE-mediated degranulation of immune effector cells, such as mast cells and basophils, resulting in the rapid manifestation of symptoms. Food allergen-derived epitopes attach to IgE molecules bound to FcεRI receptors on the surface of these effector cells; epitope-specific crosslinking of IgE-bound receptors then occurs, leading to the release of preformed histamine and other inflammatory mediators of the immediate allergic reaction (Stone et al., J. Allergy Clin. Immunol. 2010; 125:S73-S80). After this immediate phase of the response, the de novo production of leukotrienes, platelet activating factor, and cytokines such as interleukin-4 (IL-4), IL-5, and IL-13 maintains allergic inflammation (Stone et al., supra). Gastrointestinal manifestations can include oral tingling, pruritus and/or swelling, as well as nausea, abdominal pain, and/or vomiting. Respiratory effects include wheezing and/or airway inflammation. Skin manifestations include flushing, urticaria, angioedema, and/or pruritus. Systemic responses may also occur, such as hypotension due to fluid leakage from the vasculature and/or hypothermia. Anaphylaxis is a serious allergic reaction that involves multiple organ systems and can rapidly become life-threatening (Yu et al., supra).

Mixed food allergies are characterized by both IgE-dependent and IgE-independent pathways. Atopic manifestations arising from IgE-independent factors include, for example, delayed food-allergy-associated atopic dermatitis caused by the action of T helper 2 (Th2) cells, and eosinophilic gastrointestinal disorders, such as eosinophilic oesophagitis (EoE), which are often triggered by milk allergens and caused by the eosinophilic infiltration of tissues (Zuo L., Rothenberg M. E., Immunol. Allergy Clin. North Am. 2007; 27:443-455; Simon et al., Allergy, 2016; 71:611-620; and Yu et al., supra).

Food allergies not mediated by IgE typically affect the gastrointestinal tract, rather than the skin and respiratory tracts. For example, allergen-specific T cells are thought to have roles in the largely unknown etiologies of food protein-induced enterocolitis syndrome (FPIES), food protein-induced proctocolitis (FPIP), and food protein enteropathy (FPE) (Nowak-Wegrzyn et al., J. Allergy Clin. Immunol. 2015; 135:1114-1124). FPIES, FPIP and FPE primarily involve infants and toddlers who are allergic to cow's milk, and they commonly resolve after one to five years.

The immune system normally develops tolerance to food proteins, at least in part due to the actions of CD4+ regulatory T cells. Food allergy develops when the immune system mounts a T helper 2 (Th2) cell-mediated response against food epitopes. Th2 cell sensitization may occur initially at the skin, rather than in the gastrointestinal tract. Inflammatory cytokines released by the skin epithelium, including IL-25, IL-33 and thymic stromal lymphopoietin (TSLP), act on dendritic cells (DCs) and other cells to shift the immune response towards TH2 cell-related allergic responses, rather than tolerogenic responses (Paul W. E., Zhu J., Nat. Rev. Immunol. 2010; 10:225-235). In this respect, TSLP may promote dendritic cell (DC) differentiation into a Th2 cell-promoting phenotype (Divekar R, Kita H., Curr. Opin. Allergy Clin. Immunol. 2015; 15:98-103; and Ito et al., J. Exp. Med. 2005; 202:1213-1223). For example, OX40L may be upregulated in DCs that promote Th2 cell differentiation of naive CD4+ T cells (Ito et al., supra). IL-25 secretion by epithelial tuft cells also may aid the expansion of type 2 innate lymphoid cell (ILC2) populations (Klose, C. S. and Artis, D., Nat. Immunol. 2016; 17:765-774), which together with Th2 cells secrete cytokines that promote the Th2 cell-mediated immune response (Mirchandani et al., J. Immunol. 2014; 192:2442-2448), which includes tissue eosinophil accumulation and IgE class-switching by B cells (Stone et al.). TH9 cells also contribute to the allergic immune response by increasing tissue mast cell accumulation (Sehra et al., J. Allergy Clin. Immunol. 2015; 136:433-440), and IL-4-mediated signaling may convert regulatory T cells into TH2 cells (Noval et al., Immunity. 2015; 42:512-523). The roles of follicular T cells, tissue-resident T cells, CD8+ T cells and γδ T cells remain to be determined. The mechanisms by which a TH2 cell-mediated response of cutaneous origin can reach the gut are unclear. One possibility is that DCs in the skin may secrete retinoic acid, which induces allergen-specific T cells to express gut-homing markers as they differentiate down the TH2 cell pathway (Hammerschmidt et al., J. Clin. Invest. 2011; 121:3051-3061; Yu et al., supra).

### Compositions

The present disclosure provides methods and compositions for the stimulation of immune responses and for inhibiting, treating, or preventing an allergic disease, particularly food allergies (e.g., airway and/or gastrointestinal inflammation). For example, the disclosure provides a method of inhibiting an allergic reaction to two or more food allergens in a subject, which comprises administering to the subject a composition (e.g., a vaccine) comprising a nanoemulsion and at least one of the two or more food allergens. In some embodiments, the subject exhibits sensitivity to the two or more (e.g., 2, 3, 4, 5, 6, 7, 8, 9, 10, or more) food allergens. The composition may comprise any food allergen or combination of food allergens. Numerous food allergens are known in the art and are present in, for example, milk (e.g., caseins and the whey proteins alpha-lactalbumin and beta-lactoglobulin), eggs (e.g., ovalbumin, ovomucoid, ovotransferrin, lysozyme), tree nuts (e.g., seed storage proteins (vicilins, legumins, albumins), plant defense related proteins and profilins), peanuts (e.g., albumins, globulins, prolamins), soy (e.g., albumins, globulins, prolamins), fish (e.g., parvalbumin), wheat (e.g., gluten), sesame, crustacean shellfish (e.g., tropomyosin), oats, rice, beef, chicken, or other food allergen known in the art (see, e.g., Adkinson et al. (eds)., Middleton's Allergy: Principles and Practice, 8^{th} Edition, Elsevier (2013)). It will be appreciated that a whole allergen may contain more than one allergenic protein, that is, it may contain a plurality of individual allergen components. Thus, the compositions described herein may comprise one or more of the plurality of individual allergen components of a whole food allergen source.

In some embodiments, a composition of the disclosure comprises a nanoemulsion and only a single food allergen, but inhibits an allergic reaction to two or more food allergens (i.e., induces bystander suppression of reactivity to multiple food allergens). In other embodiments, the composition comprises each food allergen against which a subject exhibits sensitivity.

In other embodiments, the disclosure provides methods of inhibiting, reducing and/or ameliorating an allergic reaction to a whole food allergen comprising a plurality of individual allergen components in a subject, the methods comprising administering to the subject a composition comprising a nanoemulsion and at least one of the plurality of individual allergen components. In some embodiments, the composition is a vaccine. The disclosure is not limited by the number of individual allergen components of the whole food allergen utilized in the composition. In some embodiments, the composition comprises a single allergen of the plurality of individual allergen components. In other embodiments, the composition comprises two, three, four or more separate allergens of the plurality of individual allergen components. One non-limiting example of a composition useful for inhibiting, reducing and/or ameliorating an allergic reaction to a whole food allergen (e.g., peanut) is a composition comprising nanoemulsion and one or more of ara h 1, ara h 2, ara h 3, and ara h 6. An allergen utilized in the compositions and methods of the disclosure may be a purified allergen (e.g., purified protein) or a recombinant allergen (e.g., a recombinant protein).

A nanoemulsion may be mixed with an allergen (e.g., a whole food allergen or one or more, but less than all, individual allergen components of a whole food allergen comprising a plurality of individual allergen components), allergenic substance, or other material that causes an allergic response. In some embodiments, the nanoemulsion comprises (a) a poloxamer surfactant or polysorbate surfactant; (b) an organic solvent; (c) a halogen-containing compound; (d) oil, and (e) water. In this regard, the nanoemulsion comprises an aqueous phase, such as, for example, water (e.g., distilled water, purified water, water for injection, de-ionized water, tap water, etc.) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments, the aqueous phase comprises phosphate buffered saline (PBS). The aqueous phase may further be sterile and pyrogen free.

The nanoemulsion may comprise any suitable organic solvent. Suitable organic solvents include, but are not limited to, C₁-C₁₂ alcohol, diol, triol, dialkyl phosphate, tri-alkyl phosphate (e.g., tri-n-butyl phosphate), semi-synthetic derivatives thereof, and combinations thereof. In one aspect, the organic solvent is an alcohol chosen from a nonpolar solvent, a polar solvent, a protic solvent, or an aprotic solvent. Other suitable organic solvents for the nanoemulsion include, but are not limited to, ethanol, methanol, isopropyl alcohol, propanol, octanol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, polyethylene glycol, an organic phosphate based solvent, semi-synthetic derivatives thereof, and any combination thereof.

The oil phase may be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof. Examples of suitable oils that may be used in the nanoemulsion include mineral oil, squalene oil, flavor oils, silicon oil, essential oils, water insoluble vitamins, isopropyl stearate, butyl stearate, octyl palmitate, cetyl palmitate, tridecyl behenate, diisopropyl adipate, dioctyl sebacate, menthyl anthranhilate, cetyl octanoate, octyl salicylate, isopropyl myristate, neopentyl glycol dicarpate cetols, ceraphyls, decyl oleate, diisopropyl adipate, C₁₂₋₁₅ alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, canola oil, chile oil, coconut oil, corn oil, cottonseed oil, flaxseed oil, grape seed oil, mustard oil, olive oil, palm oil, palm kernel oil, peanut oil, pine seed oil, poppy seed oil, pumpkin seed oil, rice bran oil, safflower oil, tea oil, truffle oil, vegetable oil, apricot (kernel) oil, jojoba oil (Simmondsia chinensis seed oil), macadamia oil, wheat germ oil, almond oil, rapeseed oil, gourd oil, soybean oil, sesame oil, hazelnut oil, maize oil, sunflower oil, hemp oil, bois oil, kuki nut oil, avocado oil, walnut oil, fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, marhoram flower oil, orange flower oil, rose flower oil, ylang-ylang flower oil, vark oil, cassia bark oil, cinnamon bark oil, sassafras bark oil, wood oil, camphor wood oil, cedar wood oil, rosewood oil, sandalwood oil, rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, oleic acid, linoleic acid, oleyl alcohol, isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organo-modified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, or combinations thereof. In certain embodiments, the volatile oil in the silicone component is different than the oil in the oil phase.

The surfactant in the nanoemulsion may be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant. Exemplary useful surfactants are described in, e.g., Applied Surfactants: Principles and Applications, Tharwat F. Tadros, WILEY-VCH Verlag GmbH & Co. KGaA, Weinheim (2005)). In other embodiments, the surfactant may be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Specific examples of suitable surfactants include ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, ethylene oxide-propylene oxide block copolymers, and tetra-functional block copolymers based on ethylene oxide and propylene oxide, glyceryl monoesters, glyceryl caprate, glyceryl caprylate, glyceryl cocate, glyceryl erucate, glyceryl hydroxysterate, glyceryl isostearate, glyceryl lanolate, glyceryl laurate, glyceryl linolate, glyceryl myristate, glyceryl oleate, glyceryl PABA, glyceryl palmitate, glyceryl ricinoleate, glyceryl stearate, glyceryl thighlycolate, glyceryl dilaurate, glyceryl dioleate, glyceryl dimyristate, glyceryl disterate, glyceryl sesuioleate, glyceryl stearate lactate, polyoxyethylene cetyl/stearyl ether, polyoxyethylene cholesterol ether, polyoxyethylene laurate or dilaurate, polyoxyethylene stearate or distearate, polyoxyethylene fatty ethers, polyoxyethylene lauryl ether, polyoxyethylene stearyl ether, polyoxyethylene myristyl ether, a steroid, cholesterol, betasitosterol, bisabolol, fatty acid esters of alcohols, isopropyl myristate, aliphati-isopropyl n-butyrate, isopropyl n-hexanoate, isopropyl n-decanoate, isoproppyl palmitate, octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In other embodiments, the surfactant may be a polyoxyethylene fatty ether having a polyoxyethylene head group ranging from about 2 to about 100 groups, or an alkoxylated alcohol having the structure R₅—(OCH₂CH₂)y-OH, wherein R₅ is a branched or unbranched alkyl group having from about 6 to about 22 carbon atoms and y is between about 4 and about 100, preferably between about 10 and about 100. Preferably, the alkoxylated alcohol is the species wherein R₅ is a lauryl group and y has an average value of 23.

In other embodiments, the surfactant may be an alkoxylated alcohol which is an ethoxylated derivative of lanolin alcohol. For example, the ethoxylated derivative of lanolin alcohol may be laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis[imidazoyl carbonyl]), BRIJ 35, BRIJ 56, BRIJ 72, BRIJ 76, BRIJ 92V, BRIJ 97, BRIJ 58P, CREMOPHOR, EL, decaethylene glycol monododecyl ether, N-decanoyl-N-methylglucamine, n-decyl alpha-D-glucopyranoside, decyl beta-D-maltopyranoside, n-dodecanoyl-N-methylglucamide, n-dodecyl alpha-D-maltoside, n-dodecyl beta-D-maltoside, n-dodecyl beta-D-maltoside, heptaethylene glycol monodecyl ether, heptaethylene glycol monododecyl ether, heptaethylene glycol monotetradecyl ether, n-hexadecyl beta-D-maltoside, hexaethylene glycol monododecyl ether, hexaethylene glycol monohexadecyl ether, hexaethylene glycol monooctadecyl ether, hexaethylene glycol monotetradecyl ether, igepal CA-630, igepal CA-630, Methyl-6-O—(N-heptylcarbamoyl)-alpha-D-glucopyranoside, nonaethylene glycol monododecyl ether, N-nonanoyl-N-methylglucamine, N-nonanoyl-N-methylglucamine, octaethylene glycol monodecyl ether, octaethylene glycol monododecyl ether, octaethylene glycol monohexadecyl ether, octaethylene glycol monooctadecyl ether, octaethylene glycol monotetradecyl ether, octyl-beta-D-glucopyranoside, pentaethylene glycol monodecyl ether, pentaethylene glycol monododecyl ether, pentaethylene glycol monohexadecyl ether, pentaethylene glycol monohexyl ether, pentaethylene glycol monooctadecyl ether, pentaethylene glycol monooctyl ether, polyethylene glycol diglycidyl ether, polyethylene glycol ether W-1, polyoxyethylene 10 tridecyl ether, polyoxyethylene 100 stearate, polyoxyethylene 20 isohexadecyl ether, polyoxyethylene 20 oleyl ether, polyoxyethylene 40 stearate, polyoxyethylene 50 stearate, polyoxyethylene 8 stearate, polyoxyethylene bis(imidazolyl carbonyl), polyoxyethylene 25 propylene glycol stearate, Saponin from Quillaja bark, SPAN 20, SPAN 40, SPAN 60, SPAN 65, SPAN 80, SPAN 85, tergitol, type 15-S-12, tergitol, type 15-S-30, tergitol, type 15-S-5, tergitol, type 15-S-7, tergitol, Type 15-S-9, tergitol, type NP-10, tergitol, type NP-4, tergitol, type NP-40, tergitol, type NP-7, tergitol, type NP-9, tergitol, tergitol, type TMN-10, tergitol, type TMN-6, tetradecyl-beta-D-maltoside, tetraethylene glycol monodecyl ether, tetraethylene glycol monododecyl ether, tetraethylene glycol monotetradecyl ether, triethylene glycol monodecyl ether, triethylene glycol monododecyl ether, triethylene glycol monohexadecyl ether, triethylene glycol monooctyl ether, triethylene glycol monotetradecyl ether, triton CF-21, triton CF-32, triton DF-12, triton DF-16, triton GR-5M, triton QS-15, triton QS-44, triton X-100, triton X-102, triton X-15, triton X-151, triton X-200, triton X-207, TRITON X-100, TRITON X-114, TRITON X-165, TRITON X-305, TRITON X-405, TRITON X-45, TRITON X-705-70, TWEEN 20, TWEEN 21, TWEEN 40, TWEEN 60, TWEEN 61, TWEEN 65, TWEEN 80, TWEEN 81, TWEEN 85, tyloxapol, n-undecyl beta-D-glucopyranoside, semi-synthetic derivatives thereof, or combinations thereof.

In a specific embodiment, the nonionic surfactant may be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, poloxamers are used in the formulation of skin cleansers, bath products, shampoos, hair conditioners, mouthwashes, eye makeup remover, and other skin and hair products. Examples of poloxamers include, but are not limited to, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, and poloxamer 182 dibenzoate.

In another embodiment, the nonionic surfactant may be a polysorbate surfactant, such as polysorbate 20 or polysorbate 80. For example, polysorbate 80 may be included in the nanoemulsion at a concentration of about 0.01% to about 5.0% (e.g., about 0.05%, about 0.08%, about 0.1%, about 0.5%, about 1.0%, about 1.5%, about 2.0%, about 2.5%, about 3.0%, about 3.5%, about 4.0%, or about 4.5%). In one embodiment, polysorbate 80 is included in the nanoemulsion at a concentration of about 0.1% to about 3% (e.g., about 0.3%, about 0.4%, about 0.6%, about 0.9%, about 1.2%, about 1.4%, about 1.6%, about 1.8%, about 2.1%, about 2.3%, about 2.5%, about 2.7%, or about 2.9%).

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, benzalkonium chloride, benzalkonium chloride, benzyldimethylhexadecylammonium chloride, benzyldimethyltetradecylammonium chloride, benzyldodecyldimethylammonium bromide, benzyltrimethylammonium tetrachloroiodate, dimethyldioctadecylammonium bromide, dodecylethyldimethylammonium bromide, dodecyltrimethylammonium bromide, dodecyltrimethylammonium bromide, ethylhexadecyldimethylammonium bromide, Girard's reagent T, hexadecyltrimethylammonium bromide, hexadecyltrimethylammonium bromide, N,N′,N′-polyoxyethylene(10)-N-tallow-1,3-diaminopropane, thonzonium bromide, trimethyl(tetradecyl)ammonium bromide, 1,3,5-Triazine-1,3,5(2H,4H,6H)-triethanol, 1-decanaminium, N-decyl-N,N-dimethyl-, chloride, didecyl dimethyl ammonium chloride, 2-(2-(p-(diisobutyl)cresosxy)ethoxy)ethyl dimethyl benzyl ammonium chloride, 2-(2-(p-(diisobutyl)phenoxy)ethoxy)ethyl dimethyl benzyl ammonium chloride, alkyl 1 or 3 benzyl-1-(2-hydroxethyl)-2-imidazolinium chloride, alkyl bis(2-hydroxyethyl) benzyl ammonium chloride, alkyl demethyl benzyl ammonium chloride, alkyl dimethyl 3,4-dichlorobenzyl ammonium chloride (100% O12), alkyl dimethyl 3,4-dichlorobenzyl ammonium chloride (50% O14, 40% C12, 10% O16), alkyl dimethyl 3,4-dichlorobenzyl ammonium chloride (55% C14, 23% C12, 20% O16), alkyl dimethyl benzyl ammonium chloride, alkyl dimethyl benzyl ammonium chloride (100% O14), alkyl dimethyl benzyl ammonium chloride (100% C16), Alkyl dimethyl benzyl ammonium chloride (41% C14, 28% C12), alkyl dimethyl benzyl ammonium chloride (47% C12, 18% C14), alkyl dimethyl benzyl ammonium chloride (55% C16, 20% C14), alkyl dimethyl benzyl ammonium chloride (58% C14, 28% C16), alkyl dimethyl benzyl ammonium chloride (60% C14, 25% C12), alkyl dimethyl benzyl ammonium chloride (61% C11, 23% C14), alkyl dimethyl benzyl ammonium chloride (61% C12, 23% C14), alkyl dimethyl benzyl ammonium chloride (65% C12, 25% C14), alkyl dimethyl benzyl ammonium chloride (67% C12, 24% C14), alkyl dimethyl benzyl ammonium chloride (67% C12, 25% C14), alkyl dimethyl benzyl ammonium chloride (90% C14, 5% C12), alkyl dimethyl benzyl ammonium chloride (93% C14, 4% C12), alkyl dimethyl benzyl ammonium chloride (95% C16, 5% C18), alkyl dimethyl benzyl ammonium chloride, alkyl didecyl dimethyl ammonium chloride, alkyl dimethyl benzyl ammonium chloride, alkyl dimethyl benzyl ammonium chloride (C12-16), alkyl dimethyl benzyl ammonium chloride (C12-18), alkyl dimethyl benzyl ammonium chloride, dialkyl dimethyl benzyl ammonium chloride, alkyl dimethyl dimethybenzyl ammonium chloride, alkyl dimethyl ethyl ammonium bromide (90% C14, 5% C16, 5% C12), alkyl dimethyl ethyl ammonium bromide (mixed alkyl and alkenyl groups as in the fatty acids of soybean oil), alkyl dimethyl ethylbenzyl ammonium chloride, alkyl dimethyl ethylbenzyl ammonium chloride (60% C14), alkyl dimethyl isopropylbenzyl ammonium chloride (50% C12, 30% C14, 17% C16, 3% C18), alkyl trimethyl ammonium chloride (58% C18, 40% C16, 1% C14, 1% C12), Alkyl trimethyl ammonium chloride (90% C18, 10% C16), alkyldimethyl-(ethylbenzyl) ammonium chloride (C12-18), di-(C8-10)-alkyl dimethyl ammonium chlorides, dialkyl dimethyl ammonium chloride, dialkyl methyl benzyl ammonium chloride, didecyl dimethyl ammonium chloride, diisodecyl dimethyl ammonium chloride, dioctyl dimethyl ammonium chloride, dodecyl bis(2-hydroxyethyl) octyl hydrogen ammonium chloride, dodecyl dimethyl benzyl ammonium chloride, dodecylcarbamoyl methyl dimethyl benzyl ammonium chloride, heptadecyl hydroxyethylimidazolinium chloride, hexahydro-1,3,5-tris(2-hydroxyethyl)-s-triazine, hexahydro-1,3,5-tris(2-hydroxyethyl)-s-triazine, myristalkonium chloride (and) Quat RNIUM 14, N,N-dimethyl-2-hydroxypropylammonium chloride polymer, n-Tetradecyl dimethyl benzyl ammonium chloride monohydrate, octyl decyl dimethyl ammonium chloride, octyl dodecyl dimethyl ammonium chloride, octyphenoxyethoxyethyl dimethyl benzyl ammonium chloride, oxydiethylenebis(alkyl dimethyl ammonium chloride), quaternary ammonium compounds, dicoco alkyldimethyl, chloride, trimethoxysily propyl dimethyl octadecyl ammonium chloride, trimethoxysilyl quats, trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some embodiments, cationic halogen-containing compounds include, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen-containing compound is CPC, although the compositions of the present disclosure are not limited to formulation with an particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, dehydrocholic acid, deoxycholic acid, deoxycholic acid, deoxycholic acid methyl ester, digitonin, digitoxigenin, N,N-dimethyldodecylamine N-oxide, docusate sodium salt, glycochenodeoxycholic acid sodium salt, glycocholic acid hydrate, synthetic, glycocholic acid sodium salt hydrate, synthetic, glycodeoxycholic acid monohydrate, Glycodeoxycholic acid sodium salt, glycodeoxycholic acid sodium salt, glycolithocholic acid 3-sulfate disodium salt, glycolithocholic acid ethyl ester, N-lauroylsarcosine sodium salt, N-lauroylsarcosine solution, N-lauroylsarcosine solution, lithium dodecyl sulfate, lithium dodecyl sulfate, lithium dodecyl sulfate, lugol solution, Niaproof 4, yype 4, 1-Octanesulfonic acid sodium salt, sodium 1-butanesulfonate, sodium 1-decanesulfonate, sodium 1-decanesulfonate, sodium 1-dodecanesulfonate, sodium 1-heptanesulfonate anhydrous, sodium 1-heptanesulfonate anhydrous, sodium 1-nonanesulfonate, sodium 1-propanesulfonate monohydrate, sodium 2-bromoethanesulfonate, sodium cholate hydrate, sodium choleate, sodium deoxycholate, sodium deoxycholate monohydrate, sodium dodecyl sulfate, sodium hexanesulfonate anhydrous, sodium octyl sulfate, sodium pentanesulfonate anhydrous, sodium taurocholate, taurochenodeoxycholic acid sodium salt, taurodeoxycholic acid sodium salt monohydrate, taurohyodeoxycholic acid sodium salt hydrate, taurolithocholic acid 3-sulfate disodium salt, tauroursodeoxycholic acid sodium salt, TRIZMA dodecyl sulfate, TWEEN 80, ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(decyldimethylammonio)propanesulfonate inner salt, 3-dodecyldimethyl-ammonio)propanesulfonate inner salt, SigmaUltra, 3-(dodecyldimethylammonio)propanesulfonate inner salt, 3-(N,N-dimethylmyristylammonio)propanesulfonate, 3-(N,N-dimethylocatdecylammonio)propanesulfonate, 3-(N,N-dimethyloctyl-ammonio)propanesulfonate inner salt, 3-(N,N-dimethylpalmitylammonio)-propanesulfonate, semi-synthetic derivatives thereof, and combinations thereof.

In some embodiments, the nanoemulsion comprises a cationic surfactant, which can be cetylpyridinium chloride (CPC). When the nanoemulsion comprises a cationic surfactant, the concentration of the cationic surfactant desirably is less than about 5.0% and greater than about 0.001%. For example, the concentration of the cationic surfactant may be less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. The concentration of the cationic agent in the nanoemulsion desirably is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%.

Alternatively, the nanoemulsion may comprise at least one cationic surfactant and at least one non-cationic surfactant. The non-cationic surfactant may be a nonionic surfactant, such as a polysorbate (Tween) (e.g., polysorbate 80 or polysorbate 20). In one embodiment, the concentration of the non-ionic surfactant is about 0.01% to about 5.0%, e.g., about 0.1% to about 3%, and the concentration of the cationic surfactant is about 0.01% to about 2%.

In certain embodiments, the nanoemulsion further comprises a halogen-containing compound, such as a cationic halogen-containing compound. The present disclosure is not limited to a particular cationic halogen-containing compound. A variety of cationic halogen-containing compounds may be included in the nanoemulsion, such as, for example, cetylpyridinium halides, cetyltrimethylammonium halides, cetyldimethylethylammonium halides, cetyldimethylbenzylammonium halides, cetyltributylphosphonium halides, dodecyltrimethylammonium halides, and tetradecyltrimethylammonium halides. The disclosed nanoemulsion composition also is not limited to a particular halide. A variety of halides may be included in the nanoemulsion composition, such as, for example, chloride, fluoride, bromide, and iodide.

The nanoemulsion may further comprise a quaternary ammonium-containing compound. Suitable quaternary ammonium-containing compounds that may be incorporated in the nanoemulsion include, but are not limited to, alkyl dimethyl benzyl ammonium chloride, dialkyl dimethyl ammonium chloride, n-alkyl dimethyl benzyl ammonium chloride, n-alkyl dimethyl ethylbenzyl ammonium chloride, dialkyl dimethyl ammonium chloride, and n-alkyl dimethyl benzyl ammonium chloride.

In other embodiments, the composition may comprise one or more adjuvants that promote a Th1 immune response, which are referred to as “Th1-polarizing adjuvants.” Examples of Th1-polarizing adjuvants include, but are not limited to, monophosphoryl lipid A (MPL), QUIL-A®, dimethyl dioctadecyl ammonium bromide (DDA), lipopolysaccharide (LPS), and unmethylated CpG oligonucleoside (CpG). Both LPS and CpG activate DCs via toll-like receptors, TLR4 and TLR9, respectively.

The present composition may comprise compounds or components in addition those described above. Such additional compounds include, but are not limited to, one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified composition comprising a nanoemulsion, or the additional compounds can be added to the original mixture to be emulsified. In certain embodiments, one or more additional compounds are admixed into an existing immunogenic composition immediately prior to its use.

Suitable preservatives that can be employed in the composition include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, chlorphenesin (3-(-4-chloropheoxy)-propane-1,2-diol), Kathon C G (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), Killitol (7.5% chlorphenesin and 7.5% methyl parabens), semi-synthetic derivatives thereof, and combinations thereof.

The disclosed composition may further comprise at least one pH adjuster, such as, for example, diethyanolamine, lactic acid, monoethanolamine, triethylanolamine, sodium hydroxide, sodium phosphate, semi-synthetic derivatives thereof, and combinations thereof.

The disclosed composition may further comprise a chelating agent. In one embodiment, the chelating agent may be present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol.

The composition may further comprise a buffering agent, such as a pharmaceutically acceptable buffering agent. Examples of buffering agents include, but are not limited to, 2-amino-2-methyl-1,3-propanediol, 99.5% (NT), 2-amino-2-methyl-1-propanol, ≥99.0% (GC), L-(+)-tartaric acid, ≥99.5% (T), ACES, ≥99.5% (T), ADA, ≥99.0% (T), acetic acid, ≥99.5% (GC/T), acetic acid, for luminescence, ≥99.5% (GC/T), ammonium acetate solution, for molecular biology, about 5 M in H₂O, ammonium acetate, for luminescence, ≥99.0% (calc. on dry substance, T), ammonium bicarbonate, 99.5% (T), ammonium citrate dibasic, 99.0% (T), ammonium formate solution, 10 M in H₂O, ammonium formate, 99.0% (calc. based on dry substance, NT), ammonium oxalate monohydrate, ≥99.5% (RT), ammonium phosphate dibasic solution, 2.5 M in H₂O, ammonium phosphate dibasic, ≥99.0% (T), ammonium phosphate monobasic solution, 2.5 M in H₂O, ammonium phosphate monobasic, ≥99.5% (T), ammonium sodium phosphate dibasic tetrahydrate, ≥99.5% (NT), ammonium sulfate solution, for molecular biology, 3.2 M in H₂O, ammonium tartrate dibasic solution, 2 M in H₂O (colorless solution at 20.degree. C.), ammonium tartrate dibasic, ≥99.5% (T), BES buffered saline, for molecular biology, 2.times. concentrate, BES, ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), bicarbonate buffer solution, ≥0.1 M Na₂CO₃, ≥0.2 M NaHCO₃, boric acid, ≥99.5% (T), boric acid, for molecular biology, ≥99.5% (T), CAPS, ≥99.0% (TLC), CHES, ≥99.5% (T), calcium acetate hydrate, ≥99.0% (calc. on dried material, KT), calcium carbonate, precipitated, 99.0% (KT), calcium citrate tribasic tetrahydrate, 98.0% (calc. on dry substance, KT), citrate concentrated solution, for molecular biology, 1 M in H₂O, citric acid, anhydrous, ≥99.5% (T), citric acid, for luminescence, anhydrous, ≥99.5% (T), diethanolamine, ≥99.5% (GC), EPPS, 99.0% (T), ethylenediaminetetraacetic acid disodium salt dihydrate, for molecular biology, ≥99.0% (T), formic acid solution, 1.0 M in H₂O, Gly-Gly-Gly, 99.0% (NT), Gly-Gly, ≥99.5% (NT), glycine, ≥99.0% (NT), glycine, for luminescence, ≥99.0% (NT), glycine, for molecular biology, ≥99.0% (NT), HEPES buffered saline, for molecular biology, 2.times. concentrate, HEPES, ≥99.5% (T), HEPES, for molecular biology, ≥99.5% (T), imidazole buffer solution, 1 M in H₂O, imidazole, ≥99.5% (GC), imidazole, for luminescence, ≥99.5% (GC), imidazole, for molecular biology, ≥99.5% (GC), lipoprotein refolding buffer, lithium acetate dihydrate, >99.0% (NT), lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), magnesium acetate solution, for molecular biology, about 1 M in H₂O, magnesium acetate tetrahydrate, ≥99.0% (KT), magnesium citrate tribasic nonahydrate, ≥98.0% (calc. based on dry substance, KT), magnesium formate solution, 0.5 M in H₂O, magnesium phosphate dibasic trihydrate, ≥98.0% (KT), neutralization solution for the in situ hybridization for in situ hybridization, for molecular biology, oxalic acid dihydrate, ≥99.5% (RT), PIPES, ≥99.5% (T), PIPES, for molecular biology, ≥99.5% (T), phosphate buffered saline, solution (autoclaved), phosphate buffered saline, washing buffer for peroxidase conjugates in Western blotting, 10 times concentrate, piperazine, anhydrous, ≥99.0% (T), potassium D-tartrate monobasic, 99.0% (T), potassium acetate solution, for molecular biology, potassium acetate solution, for molecular biology, 5 M in H₂O, potassium acetate solution, for molecular biology, about 1 M in H₂O, potassium acetate, ≥99.0% (NT), potassium acetate, for luminescence, 99.0% (NT), potassium acetate, for molecular biology, ≥99.0% (NT), potassium bicarbonate, ≥99.5% (T), potassium carbonate, anhydrous, ≥99.0% (T), potassium chloride, ≥99.5% (AT), potassium citrate monobasic, 99.0% (dried material, NT), potassium citrate tribasic solution, 1 M in H₂O, potassium formate solution, 14 M in H₂O, potassium formate, ≥99.5% (NT), potassium oxalate monohydrate, ≥99.0% (RT), potassium phosphate dibasic, anhydrous, ≥99.0% (T), potassium phosphate dibasic, for luminescence, anhydrous, 99.0% (T), potassium phosphate dibasic, for molecular biology, anhydrous, ≥99.0% (T), potassium phosphate monobasic, anhydrous, ≥99.5% (T), potassium phosphate monobasic, for molecular biology, anhydrous, ≥99.5% (T), potassium phosphate tribasic monohydrate, ≥95% (T), potassium phthalate monobasic, ≥99.5% (T), potassium sodium tartrate solution, 1.5 M in H2O, potassium sodium tartrate tetrahydrate, ≥99.5% (NT), potassium tetraborate tetrahydrate, ≥99.0% (T), potassium tetraoxalate dihydrate, ≥99.5% (RT), propionic acid solution, 1.0 M in H₂O, STE buffer solution, for molecular biology, pH 7.8, STET buffer solution, for molecular biology, pH 8.0, sodium 5,5-diethylbarbiturate, ≥99.5% (NT), sodium acetate solution, for molecular biology, 3 M in H₂O, sodium acetate trihydrate, 99.5% (NT), sodium acetate, anhydrous, ≥99.0% (NT), sodium acetate, for luminescence, anhydrous, ≥99.0% (NT), sodium acetate, for molecular biology, anhydrous, ≥99.0% (NT), sodium bicarbonate, ≥99.5% (T), sodium bitartrate monohydrate, ≥99.0% (T), sodium carbonate decahydrate, ≥99.5% (T), sodium carbonate, anhydrous, ≥99.5% (calc. on dry substance, T), sodium citrate monobasic, anhydrous, ≥99.5% (T), sodium citrate tribasic dihydrate, ≥99.0% (NT), sodium citrate tribasic dihydrate, for luminescence, ≥99.0% (NT), sodium citrate tribasic dihydrate, for molecular biology, ≥99.5% (NT), sodium formate solution, 8 M in H₂O, sodium oxalate, ≥99.5% (RT), sodium phosphate dibasic dihydrate, ≥99.0% (T), sodium phosphate dibasic dihydrate, for luminescence, 99.0% (T), sodium phosphate dibasic dihydrate, for molecular biology, ≥99.0% (T), sodium phosphate dibasic dodecahydrate, ≥99.0% (T), sodium phosphate dibasic solution, 0.5 M in H₂O, sodium phosphate dibasic, anhydrous, ≥99.5% (T), sodium phosphate dibasic, for molecular biology, ≥99.5% (T), sodium phosphate monobasic dihydrate, ≥99.0% (T), sodium phosphate monobasic dihydrate, for molecular biology, ≥99.0% (T), sodium phosphate monobasic monohydrate, for molecular biology, ≥99.5% (T), sodium phosphate monobasic solution, 5 M in H₂O, sodium pyrophosphate dibasic, 99.0% (T), sodium pyrophosphate tetrabasic decahydrate, ≥99.5% (T), sodium tartrate dibasic dihydrate, ≥99.0% (NT), sodium tartrate dibasic solution, 1.5 M in H₂O (colorless solution at 20. degree. C.), sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10. times. concentrate, TRIS acetate-EDTA buffer solution, for molecular biology, TRIS buffered saline, 10. times. concentrate, TRIS glycine SDS buffer solution, for electrophoresis, 10. times. concentrate, TRIS phosphate-EDTA buffer solution, for molecular biology, concentrate, 10. times. concentrate, Tricine, ≥99.5% (NT), Triethanolamine, ≥99.5% (GC), Triethylamine, 99.5% (GC), Triethylammonium acetate buffer, volatile buffer, −1.0 M in H₂O, Triethylammonium phosphate solution, volatile buffer, about 1.0 M in H₂O, Trimethylammonium acetate solution, volatile buffer, about 1.0 M in H₂O, Trimethylammonium phosphate solution, volatile buffer, about 1 M in H₂O, Tris-EDTA buffer solution, for molecular biology, concentrate, 100 times concentrate, Tris-EDTA buffer solution, for molecular biology, pH 7.4, Tris-EDTA buffer solution, for molecular biology, pH 8.0, TRIZMA acetate, ≥99.0% (NT), TRIZMA base, ≥99.8% (T), TRIZMA base, ≥99.8% (T), TRIZMA base, for luminescence, ≥99.8% (T), TRIZMA base, for molecular biology, ≥99.8% (T), TRIZMA carbonate, ≥98.5% (T), TRIZMA hydrochloride buffer solution, for molecular biology, pH 7.2, TRIZMA hydrochloride buffer solution, for molecular biology, pH 7.4, TRIZMA hydrochloride buffer solution, for molecular biology, pH 7.6, TRIZMA hydrochloride buffer solution, for molecular biology, pH 8.0, TRIZMA hydrochloride, ≥99.0% (AT), TRIZMA hydrochloride, for luminescence, ≥99.0% (AT), TRIZMA hydrochloride, for molecular biology, ≥99.0% (AT), and TRIZMA maleate, ≥99.5% (NT).

The composition can comprise one or more emulsifying agents to aid in the formation of the nanoemulsion. Emulsifying agents include compounds that aggregate at the oil/water interface to form a continuous membrane that prevents direct contact between two adjacent droplets. The composition may also further comprise one or more immune modulators. Examples of immune modulators include, but are not limited to, chitosan and glucan. An immune modulator can be present in the composition at any pharmaceutically acceptable amount, e.g., from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, or a range defined by any two of the foregoing values.

The composition may be formulated into pharmaceutical compositions which comprise therapeutically effective amounts of the nanoemulsion, one or more food allergens, and a pharmaceutically-acceptable carrier. The choice of carrier will be determined by the practitioner, and a variety of suitable pharmaceutically-acceptable excipients are well known in the art.

In certain embodiments, the nanoemulsion comprises (a) about 3 vol. % to about 15 vol. % (e.g., about 4 vol. %, 5 vol. %, 6 vol. %, 7 vol. %, 8 vol. %, 9 vol. %, 10 vol. %, 11 vol. %, 12 vol. %, 13 vol. %, or 14 vol. %) of a poloxamer surfactant or polysorbate surfactant; (b) about 3 vol. % to about 15 vol. % (e.g., about 4 vol. %, 5 vol. %, 6 vol. %, 7 vol. %, 8 vol. %, 9 vol. %, 10 vol. %, 11 vol. %, 12 vol. %, 13 vol. %, or 14 vol. %) of an organic solvent; (c) about 0.5 vol. % to about 1 vol. % (e.g., about 0.6 vol. %, 0.7 vol. %, 0.8 vol. %, or 0.9 vol. %) of a halogen-containing compound; (d) about 3 vol. % to about 90 vol. % (e.g., about 5 vol. %, 10 vol. %, 20 vol. %, 30 vol. %, 40 vol. %, 50 vol. %, 60 vol. %, 70 vol. %, 80 vol. %, or 85 vol. %) of an oil; and (e) about 5 vol. % to about 60 vol. % (e.g., about 10 vol. %, 20 vol. %, 30 vol. %, 40 vol. %, or 50 vol. %) of water. An exemplary nanoemulsion that may be used is designated “W805EC,” and the components of which are shown in Table 1. The mean droplet size for the W805EC nanoemulsion is about 400 nm. All of the components of the nanoemulsion are included on the FDA inactive ingredient list for Approved Drug Products.

TABLE 1
W805EC Nanoemulsion Formulation

| Component | Function |
|---|---|
| Purified water, USP | Aqueous diluent |
| Soybean oil, USP (super refined) | Hydrophobic oil (core) |
| Dehydrated alcohol, USP (anhydrous | Organic solvent |
| ethanol) | |
| Polysorbate 80, NF | Surfactant |
| Cetylpyridinium chloride, USP | Emulsifying agent |

Another exemplary nanoemulsion that may be employed in the disclosed composition is designated “60% W805EC,” the components of which are set forth in Table 2.

TABLE 2
60% W805EC Formulation

| Component | Amount (w/w %) |
|---|---|
| Purified water, USP | 54.10% |
| Soybean oil, USP (super refined) | 37.67% |
| Dehydrated alcohol, USP (anhydrous | 4.04% |
| ethanol) | |
| Polysorbate 80, NF | 3.55% |
| Cetylpyridinium chloride, USP | 0.64% |

### Methods of Preparing Nanoemulsions

Generally, nanoemulsions encompassed by the present disclosure are formed by emulsification of an oil, purified water, nonionic detergent, organic solvent, and surfactant (e.g., a cationic surfactant). In this regard, the nanoemulsion may be formed using classic emulsion forming techniques, such as those described in U.S. Pat. No. 7,767,216. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the present disclosure employ a nanoemulsion having an oil phase comprising an alcohol such as ethanol. The oil and aqueous phases can be blended using any apparatus capable of producing shear forces sufficient to form an emulsion, such as French presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452.

In one embodiment, the nanoemulsion may comprise droplets of an oily discontinuous phase dispersed in an aqueous continuous phase, such as water or phosphate buffered saline (PBS). The nanoemulsion can be produced in large quantities and be stable for many months at a broad range of temperatures. The nanoemulsion can have textures ranging from that of a semi-solid cream to that of a thin lotion and can be applied topically by any pharmaceutically acceptable method, e.g., by hand, or nasal drops/spray.

As discussed above, at least a portion of the emulsion may be in the form of lipid structures including, but not limited to, unilamellar, multilamellar, and paucliamellar lipid vesicles, micelles, and lamellar phases.

It will be appreciated that variations of the described nanoemulsions will be useful in the compositions and methods disclosed herein. To determine if a candidate nanoemulsion is suitable for use with the present disclosure, three criteria are analyzed. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable will lose their form within a day. Third, the candidate nanoemulsion should have efficacy for its intended use. For example, the emulsions of the disclosure should maintain (e.g., not decrease or diminish) and/or enhance the immunogenicity of allergen (e.g., an aeroallergen), or induce a protective immune response to a detectable level.

The nanoemulsion can be provided in many different types of containers and delivery systems. For example, in some embodiments, the nanoemulsion may be provided in a cream or other solid or semi-solid form. Alternatively, the nanoemulsion may be incorporated into hydrogel formulations. The nanoemulsion can be delivered (e.g., to a subject or customers) in any suitable container. Suitable containers can be used that provide one or more single use or multi-use dosages of the nanoemulsion for the desired application. In some embodiments, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation. These nanoemulsion-containing containers can further be packaged with instructions to form kits.

Generally, emulsion compositions disclosed herein will comprise at least 0.001% to 100%, preferably 0.01 to 90%, of emulsion per ml of liquid composition. It is envisioned that the formulations may comprise about 0.001%, about 0.0025%, about 0.005%, about 0.0075%, about 0.01%, about 0.025%, about 0.05%, about 0.075%, about 0. 1%, about 0.25%, about 0.5%, about 1.0%, about 2.5%, about 5%, about 7.5%, about 10%, about 12.5%, about 15%, about 20%, about 25%, about 30%, about 35%, about 40%, about 50%, about 55%, about 60%, about 65%, about 70%, about 75%, about 80%, about 85%, about 90%, about 95% or about 100% of emulsion per ml of liquid composition. It should be understood that a range between any two figures listed above is specifically contemplated to be encompassed within the metes and bounds of a composition of the disclosure.

In some embodiments, a nanoemulsion composition is formulated to comprise between 0.1 and 500 μg of antigen (e.g., between 0.1 and 500 μg of one or more whole food allergens, or, between 0.1 and 500 μg of one or more, but less than all, individual allergen components of a whole food allergen comprising a plurality of individual allergen components). For example, the nanoemulsion composition may contain between 0.5 μg and 50 μg (e.g., about 1 μg, about 5 μg, about 10 μg, about 20 μg, about 30 μg, or about 40 μg) of antigen, 50 μg of antigen, between 50 μg and 100 μg (e.g., about 60 μg, about 70 μg about 80 μg, or about 90 μg) of antigen, 100 μg or more (e.g., about 200 μg, about 300 μg, or about 400 μg) of antigen, or a range defined by any two of the foregoing values. However, the present disclosure is not limited to this amount of antigen. For example, in some embodiments, more than 500 μg (e.g., 600 μg, 700 μg, 800 μg, 900 μg, 1 mg, or more of antigen (e.g., one or more food allergens) is present in nanoemulsion disclosed herein (e.g., for use in administration to a subject). In some embodiments, less than 1.0 μg of antigen (e.g., 900 nanograms (ng), 800 μg, 700 μg, 600 μg, 500 ng, 400 μg, 300 μg, 200 μg, 100 μg, 50 μg, 25 μg, 10 μg, or less) is present in nanoemulsion disclosed herein for administration to a subject. As described herein, at least one of two or more food allergens is present in a nanoemulsion. In some embodiments, the composition comprises one or more whole food allergens (e.g., all of the food antigens) or one or more, but less than all, individual allergen components of a whole food allergen comprising a plurality of individual allergen components against which a subject exhibits sensitivity. In some embodiments, a subject is administered, over time, different nanoemulsion compositions that are each formulated to comprise a different amount of antigen. For example, in some aspects, a subject is initially administered a formulation comprising a first amount of antigen (e.g., 25, 50, 100, 200 or 250 μg of antigen) and at a later time point (e.g., weeks, months, years later) the subject is administered a formulation comprising a second amount of antigen (e.g., a greater amount of antigen than the first amount (e.g., 1.5 times, 2 times, 2.5 times or more than the first amount), or a lesser amount of antigen than the first amount (e.g., one-half, one-third, one-fourth, or less than the first amount). In further aspects, the subject may receive one or more subsequent administrations of a nanoemulsion composition after initially being administered a formulation comprising a first amount of antigen (e.g., wherein the subsequent administrations each contain an amount of antigen that is greater than the previously administered amount of antigen).

### Treatment Methods

The disclosure also provides a method of inhibiting an allergic reaction to two or more food allergens in a subject, which comprises administering an effective amount of a composition disclosed herein to a subject in need thereof, whereupon an allergic reaction to the two or more food allergens in the subject is inhibited. In other words, the compositions and methods described herein desirably result in the treatment of an allergy to two or more food allergens in a subject. In other embodiments, the disclosure provides a method of inhibiting, reducing and/or ameliorating an allergic reaction to a whole food allergen comprising a plurality of individual allergen components in a subject, which method comprises administering an effective amount of a composition disclosed herein (e.g., comprising a nanoemulsion and at least one of the plurality of individual allergen components) to a subject in need thereof, whereupon an allergic reaction to the whole food allergen in the subject is inhibited. In other words, the compositions and methods described herein desirably result in the treatment of an allergy to a whole food allergen comprising a plurality of individual allergen components (e.g., utilizing a composition comprising a single allergen of the plurality of individual allergen components).

As used herein, the terms “treatment,” “treating,” and the like refer to obtaining a desired pharmacologic and/or physiologic effect. In some embodiments, the effect is therapeutic, i.e., the effect partially or completely cures a disease and/or adverse symptom attributable to the disease. To this end, the disclosed methods comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the composition to elicit a desired response in the individual. For example, a therapeutically effective amount of the composition of the disclosure is an amount which decreases allergen-induced inflammation or other adverse allergic condition in a human.

Alternatively, in some embodiments, the pharmacologic and/or physiologic effect may be prophylactic, i.e., the effect completely or partially prevents, inhibits, reduces and/or ameliorates a disease or symptom thereof. In this respect, the disclosed methods comprises administering a “prophylactically effective amount” of the composition. A “prophylactically effective amount” refers to an amount effective, at dosages and for periods of time necessary, to achieve a desired prophylactic result (e.g., prevention of disease onset).

Administration of the composition comprising a nanoemulsion and at least one of two or more food allergens may treat, prevent, suppress, or inhibit any symptom or condition associated with a food allergy, such as those described herein. In some embodiments, administration of the composition desirably suppresses an anaphylactic reaction to a food allergen. Food-induced anaphylaxis is a serious allergic reaction that is rapid in onset and may cause death (Sampson et al., J Allergy Clin Immunol. 2005 March; 115(3):584-91; Nowak-Wegrzyn et al., Pediatrics. 2003 April; 111(4 Pt 1):829-35). Anaphylaxis is highly likely when any one of the following criteria are fulfilled: (1) sudden onset of an illness, with involvement of the skin, mucosal tissue, or both, and at least one of respiratory compromise or reduced blood pressure or associated symptoms of end-organ dysfunction; (2) two or more of the following that occur rapidly after exposure to a likely allergen: skin/mucosal involvement, respiratory compromise, reduced blood pressure, or gastrointestinal (GI) symptoms; and (3) reduced blood pressure after exposure to a known allergen (Simons et al., World Allergy Organ J. 2011; 4:13-37). Typically, IgE-mediated food-induced anaphylaxis is believed to involve systemic mediator release from sensitized mast cells and basophils. In some cases, such as food-dependent, exercise-induced anaphylaxis, the ability to induce reactions depends on the temporal association between food consumption and exercise, usually within two hours.

The nanoemulsion composition can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for intranasal administration. Intranasal administration includes administration via the nose, either with or without concomitant inhalation during administration. Such administration is typically through contact by the with the nasal mucosa, nasal turbinates or sinus cavity. Such administration may also include contact with the oral mucosa, bronchial mucosa, and other epithelia. The composition may be applied in a single administration or in multiple administrations.

The composition desirably is administered after exposure (or after suspected exposure or prior to impending exposure) to an allergen (e.g., two or more food allergens) to which the subject is hypersensitive. For example, the composition may be administered at least once between 0 to 30 days, between 0 to 20 days, between 0 to 15 days, or between 0 to 7 days, after the subject has been exposed to the allergen(s) to which the subject is hypersensitive. In another aspect, the composition may be administered daily for a specified time. For example, the composition may be administered daily for at least one week, at least one month, at least 3 months, 6 months, a year, or longer. In other embodiments, the composition may be administered prior to exposure to a food allergen to prevent the onset of an allergic reaction (e.g., anaphylaxis).

In some embodiments, the composition may be administered in a regimen that includes at least 1, 2, 3, 4, 5, 6, 7, 8, 9, 10, or more cycles of daily treatment. A cycle includes: (a) a period during which the composition is administered daily (e.g., 1-30 days), followed by (b) a rest period of at least one day (e.g., at least one week, 2 weeks, 3 weeks, a month, or more) in which the composition is not administered. The number of days of administration and rest can be the same or different within a cycle. Likewise, two or more consecutive cycles can have the same or a different duration. In some embodiments, the composition is administered in a regimen that includes 2 or more cycles of daily treatment, i.e., the composition is administered to the subject two or more times.

Administration of the composition described herein desirably results in the reduction or inhibition of the expression of Th2 type cytokines in the subject. Thus, in some embodiments, the disclosed compositions may be used to modulate (e.g., reduce or skew away from) Th2 immune responses (characterized by robust expression of Th2 cytokines (IL-4, IL-5, and IL-13) and IgG1) toward a balanced Th1/Th2 response (characterized by reduced IgE and Th2 response and increased IFN-gamma, TNF-alpha, IgG2a, IgG2b, IgA, IL-10, and IL-17) as a therapeutic for allergic disease, inflammatory disease, or any other disease associated with Th2 immunity.

It is believed that interferon-γ (IFN-γ) can reduce allergic disease through suppression of Th2 cells as well as effects on the innate cells and alarmins which are required for both the induction and maintenance of allergic disease. Alarmins (also referred to as “damage-associated molecular patterns (DAMPs)”) are endogenous, constitutively expressed, chemotactic, and immune activating proteins/peptides that are released as a result of degranulation, cell injury or death, or in response to immune induction. Alarmins function as intercellular signals defense by interacting with chemotactic and pattern recognition receptors (PRRs) to activate immune cells in host defense (Oppenheim J J, Yang D., Curr Opin Immunol. 2005; 17:359-365). Release of alarmins in excess by severe injuries and maximal stimulation results in a potentially lethal cytokine storm. Alarmins have been shown to participate in diverse processes including antimicrobial defense, regulation of gene expression, cellular homeostasis, wound healing, inflammation, allergy, immunity, autoimmunity, and oncogenesis. Examples of alarmins include, but are not limited to, high-mobility group box-1 (HMGB1), HMGN1, IL-1α, and IL-33, as well as heat shock proteins (HSPs), S100 proteins, ATP, and uric acid crystals (Yang et al., Immunol. Rev., 017 November; 280(1): 41-56).

Innate cytokines, including the alarmins IL-25, IL-33 and TSLP, are produced by epithelial cells and are key mediators of allergic disease (50). IL-4 and IL-13-producing type 2 innate lymphoid cells (ILC2s) have also been shown to promote experimental food allergy (51, 52). IFN-γ prevents the accumulation of these lymphoid cells in mucosal tissues by limiting the effects of alarmins required for the recruitment and maintenance of these cells (53-55). Thus, in some embodiments, administration of the nanoemulsion composition described herein may reduce expression of alarmins in the subject and/or induce expression of IFN-γ in the subject. Indeed, as demonstrated in the Examples herein, the nanoemulsion composition provided herein suppressed alarmin expression in an IFN-γ-dependent mechanism, and this correlated with suppression of allergic reactivity to bystander allergens. Thus, the present disclosure provides that local induction of IFN-γ by the nanoemulsion composition can be used to modulate the small intestine environment to suppress the allergic response (e.g., in the absence of having to eliminate allergen-specific IgE).

The following examples further illustrate the disclosure should not be construed as in any way limiting its scope.

### EXAMPLES

The following materials and methods were used in the experiments described below.

Antigen and adjuvants. Nanoemulsion adjuvant (NE) was produced by a high-speed emulsification of ultra-pure soybean oil with cetyl pyridinium chloride, Tween 80 and ethanol in water, resulting in NE droplets with an average 350-400 nm diameter (17, 29). Aluminum hydroxide (alum, alhydrogel) was purchased from InVivoGen. Peanut extract (Greer) was used for all i.p. and i.n. immunizations. For i.g. challenges, peanut flour (12% fat, light roast, Byrd Mill) was solubilized in PBS. Endotoxin-free ovalbumin (ova) was purchased from Hyglos. Endotoxin content of all vaccine components was determined by a limulus amebocyte lysate (LAL) assay (Pierce).

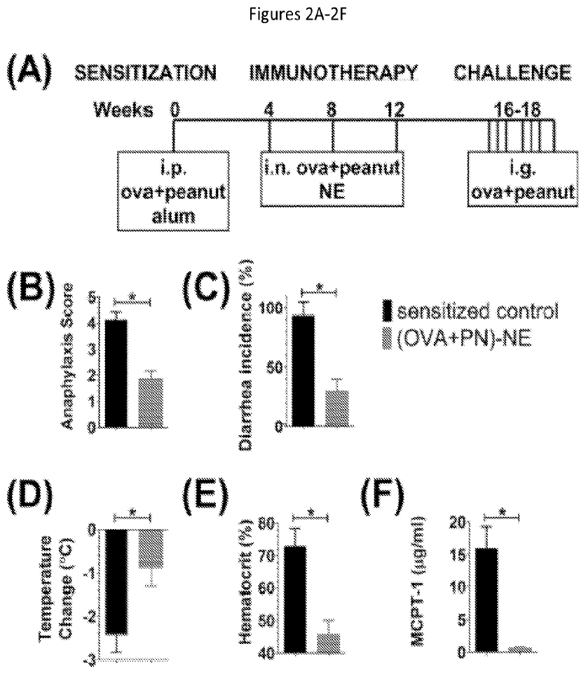

Mice and Immunizations. Specific pathogen-free BALB/c mice (females 3 weeks old) were purchased from Jackson Laboratory. Mice were 4 weeks of age at the onset of the experiment. The experimental design is shown in FIG. 2A. Allergic sensitization was induced with intraperitoneal immunizations (i.p.) of 20 μg ovalbumin and/or 20 μg peanut extract adsorbed on 1 mg alum at week 0. Intranasal (i.n.) immunizations were administered as 12 l (6 μl/nare) of a formulation containing 20 μg of ova and/or 20 μg peanut extract, or 20 μg hepatitis B surface antigen mixed with 20% NE. Allergen mixed with PBS alone served as a control. Anaphylaxis was induced by repeated oral challenge with allergen in which mice were fasted for 5-6 hours to ensure gastric emptying and then were challenged by oral gavage with 0.2 ml containing 10 mg ova and 10 mg peanut. Mice were challenged orally every other day for a total of 7 gavages (30). In IFN-γ depletion experiments, mice were injected i.p. with 0.5 mg anti-IFN-7 (XMG-6) or isotype control rat IgG1 (GL113) (31) the day before starting oral challenges and every 4 days until the final challenge. All animal procedures were performed according to the University of Michigan Institutional Animal Care and Use Committee and the National Institutes of Health guide for the care and use of laboratory animals.

Assessment of hypersensitivity reactions. Anaphylactic symptoms were evaluated for one hour following challenge with ova using the following scoring system (modified from (32, 33)): 0, no symptoms; 0.5, transient rubbing and scratching; 1, prolonged rubbing and scratching around the nose, eyes, or head; 2, puffiness around the eyes or mouth, diarrhea, piloerection, and/or decreased activity with increased respiratory rate; 3, labored respiration, wheezing, stridor, and/or cyanosis around the mouth and tail; 4, tremor, convulsion, no activity after prodding and/or moribund; 5, death. Rectal temperature was monitored for at least 60 minutes following challenge. Mice were bled 60 minutes following challenge, and serum mouse mast cell protease-1 (MCPT-1) was determined by ELISA (eBioscience). To determine hemoconcentration, blood was drawn into heparinized capillary tubes and centrifuged for 5 minutes at 10,000 rpm. Hematocrit values were calculated as the length of packed RBCs as a percentage of the total length of serum and red cells in the capillary tube.

Measurement of serum IgE. Sera were obtained by saphenous vein bleeding or by cardiac puncture post-euthanasia. Serum was separated from whole blood by centrifugation at 1500×g for 5 minutes after allowing coagulation for 30 to 60 minutes at room temperature. Serum samples were stored at −20° C. until analyzed. Ova-specific IgE antibody levels were determined by ELISA. Serially diluted serum samples were incubated on microtiter plates coated with 20 μg/mL ova. IgE antibodies were detected with alkaline phosphatase conjugated anti-mouse IgE (Rockland) and SIGMAFAST™ p-nitrophenyl phosphate substrate and quantified by measuring the optical density (OD) at 405 nm. The antibody concentrations are presented as endpoint titers defined as the reciprocal of the highest serum dilution producing an OD above background of naïve sera. The cutoff value is determined as the OD (mean+2 standard deviations) of the corresponding dilution of naive sera (34, 35).

Analysis of cytokine production. The cellular recall response was evaluated in lymphocytes isolated from mesenteric lymph nodes. Single cell lymphocyte suspensions were cultured ex vivo±ovalbumin (20 μg/ml) at 37° C. After 72 hours, cytokine secretion was measured in cell culture supernatants using Luminex Multiplex detection system (Millipore). For real-time PCR analysis, RNA was isolated from duodenum homogenates with an RNeasy mini kit (Qiagen), and cDNA was generated with a Superscript II reverse transcription kit (Invitrogen). qPCR was performed with SYBR green master mix and commercially available primer sets (Bio-Rad). Values were normalized to GAPDH and displayed as fold induction over control samples.

Lamina propria mononuclear cells isolation. Small intestine (SI, 15 cm) was dissected from the mouse and Peyer's patches were trimmed off. SI was cut longitudinally and washed with PBS thoroughly to remove ingested food. SI was incubated in a petri dish with 10 mL PBS with EDTA (5 mM) for 10 minutes on ice. Intestines were washed with PBS (no EDTA) by vortexing vigorously to remove the epithelial cells. These two steps were repeated 3-4 times until the tissue became clear. Tissue was minced finely and transferred to 8 mL digestion buffer (16 mg collagenase A (Roche) and 1.6 mg DNASe I (Roche) in RPMI (10% FBS)) and incubated at 37° C. for 30 minutes. After incubation, digested tissue was passed through a 10 ml syringe with 18G needle a few times. Liberated cells were filtered through 70 m filter. The cell suspension was washed by adding 20 mL of RPMI with 10% FBS. The cell pellet was suspended in 44% Percoll (4 mL) and loaded on 67% Percoll (3 mL) for centrifugation. A mononuclear cell gradient was created by spinning the cells down at 1800 rpm for 20 minutes at room temperature with centrifuge acceleration set at 5 and deceleration set to 0. The middle interphase of mononuclear cells was collected from the interface and washed again with RPMI (10% FBS). The obtained cells were counted and used for subsequent analysis.

Antibodies. All the antibodies used for flow cytometry were purchased from eBioscience, Biolegend and BD biosciences. For cell surface staining, a lineage cocktail consisting anti-mouse CD3 (clone 145-2C11), anti-mouse Ly-6G/Ly-6C (clone RB6-8C5), anti-mouse CD11b (clone M1/70), anti-mouse CD45R/B220 (clone RA3-6B2), and anti-mouse TER-119/Erythroid cells (clone Ter-119) was used. FITC-streptavidin was used to stain biotin labelled primary antibody cocktail. Other antibodies used were rat anti-mouse CD45, anti-mouse CD127 (clone A7R34), anti-mouse CD90.2 (clone 53-2.1), anti-mouse KLRG-1 (clone 2F1), and anti-mouse GATA 3 (clone TWAJ). FOXp3 fixation and permeabilization kit (eBioscience) was used for intracellular staining.

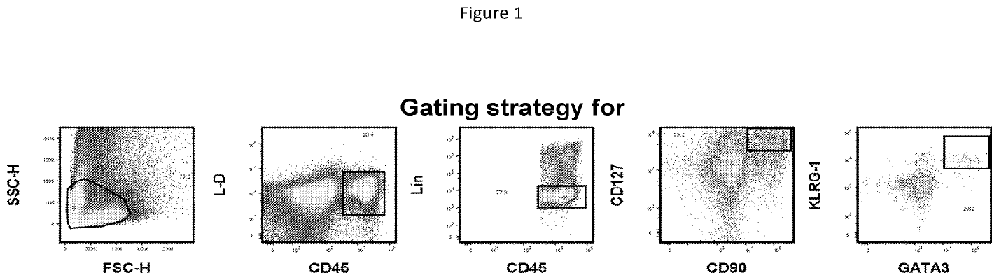

Flow cytometry. Cells were stained with lineage antibody cocktail on ice for 20 minutes followed by washing with flow staining buffer (PBS with 0.1% BSA) two times. Cells were then incubated with FITC-streptavidin antibody on ice for another 20 minutes. Cells were washed two times with flow staining buffer. Cells were then stained with live-dead ef450, CD45, CD127, CD90.2 and KLRG-1 on ice for 20 minutes followed by washing two times with flow staining buffer. Intracellular staining of GATA-3 was done using Foxp3 fixation and permeabilization kit as per the manufacturer's protocol (eBiosciences). Samples were acquired on Novocyte 3000 (Acea Biosciences) and data were analyzed using FlowJo v10.1. The gating strategy for identifying ILC2s is shown in FIG. 1.

Statistics. Statistical comparisons were assessed by the Mann-Whitney test using GraphPad Prism version 7 (GraphPad Software). The p value<0.05 was considered as significant. Results presented here are the representatives of at least two independent experiments.

### Example 1

This example demonstrates that intranasal immunization with allergens in NE adjuvant suppresses allergic reactions in polysensitized mice.

BALB/c mice were sensitized to egg and peanut at week 0 by i.p. injection of ova and peanut extract adsorbed on alum (25). Beginning four weeks after sensitization, mice were intranasally immunized three times, at 4-week intervals, with ova and peanut formulated in NE or PBS as a control. The mice were subsequently challenged orally with ova and peanut to assess protection. Sensitized control mice had profound physiological reactions to challenge as indicated by severe symptoms of anaphylactic shock, including diarrhea, labored respiration, wheezing, lack of activity when prodded, core body temperature loss of greater than 2° C., hemoconcentration, and increased mast cell degranulation (MCPT-1) (FIG. 2). The NE vaccine markedly suppressed these responses to allergen challenge. Anaphylaxis symptoms were markedly reduced to mild symptoms such as pruritus or reduced activity (FIG. 2B), and the incidence of diarrhea was reduced from 100% to 40% (FIG. 2C). Mice treated with the NE composition also were protected from hypovolemic shock and experienced minimal body temperature loss while hemoconcentration also was prevented (FIGS. 2D-2E). MCPT-1 measured in serum following challenge was used to assess mast cell degranulation. Consistent with the clinical symptoms of allergic reaction, immunized mice had significant reductions in MCPT-1, with average levels of 0.6 μg/ml compared with 16 μg/ml in sensitized control mice (FIG. 2F; p=0.0079).

### Example 2

This example demonstrates that immunization of polysensitized mice with the NE composition induces bystander suppression of allergic reactivity.

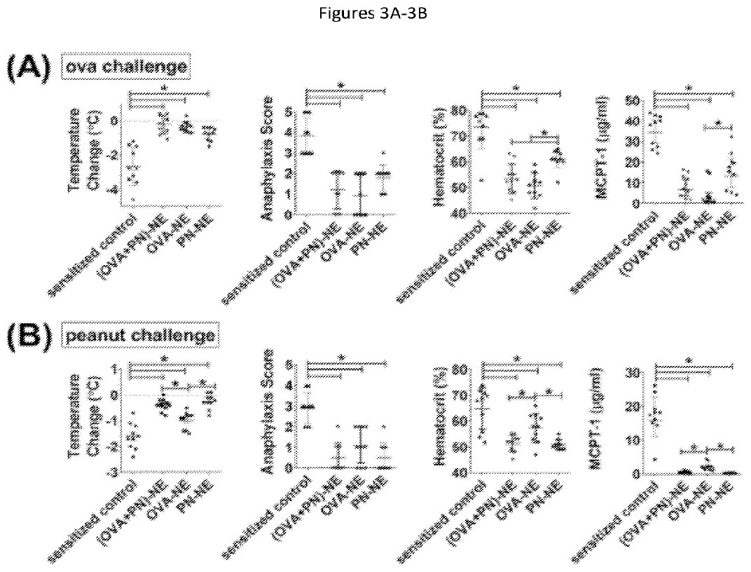

The effects of immunotherapy with NE and only one allergen on protection were assessed in polysensitized animals. Mice were again sensitized to both egg and peanut, and then were nasally treated with either ova, peanut, or both allergens formulated in NE. Mice were subsequently challenged with either ova or peanut to evaluate allergen reactivity. In general, mice were protected from allergic reaction to whatever allergen was contained in the NE composition, and protection for each allergen was similar if the mice were treated with the composition containing either a single allergen or both allergens (FIGS. 3A-3B). Surprisingly, mice that were treated with the ova-NE vaccine were also protected from reactivity to challenge with peanut, and mice treated with the peanut-NE vaccine were protected from challenge with ova. While there was a trend that this “bystander protection” was not as complete as protection induced by immunotherapy with NE and both allergens, these differences were not significant, and mice immunized with only one allergen in NE had significantly less severe allergic reactions compared with sensitized control mice that did not receive the i.n. vaccine.

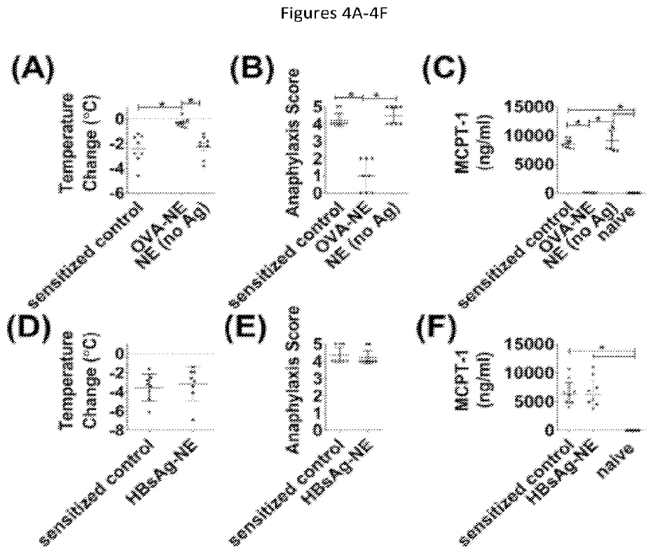

Bystander suppression of allergic reactivity required immunotherapy with NE and at least one allergen to which the mice were sensitized, as i.n. instillation of either NE alone (no allergen) or NE formulated with an unrelated antigen (hepatitis B surface antigen (HBsAg)) did not confer protection from challenge with ova (FIGS. 4A-4F). Taken together, these data indicate that immunotherapy with NE and a single allergen induces allergen-specific immune response that results in bystander suppression of allergic reactivity to other allergens.

### Example 3

This example demonstrates that intranasal immunization with NE adjuvant suppresses allergy associated Th2 responses and alarmin expression.

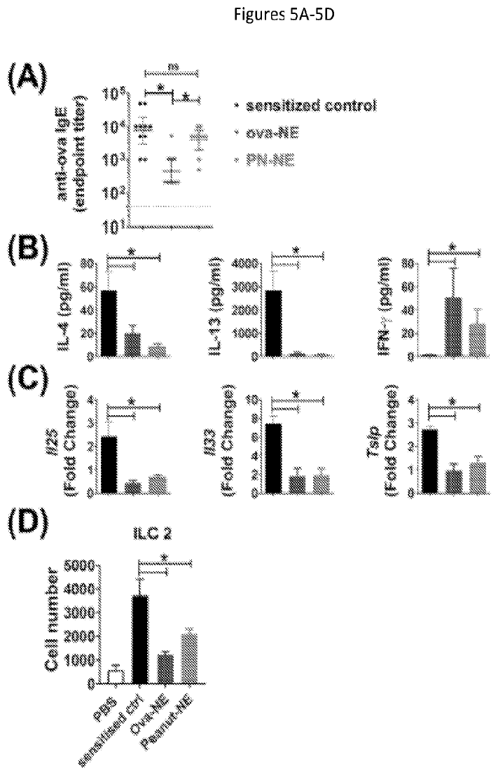

While food allergic reactions are dependent upon the presence of allergen-specific IgE, many patients with allergen-specific IgE to foods do not clinically react to those foods. In this Example, immunization with PN-NE suppressed allergic reactivity to both peanut and ova without significantly reducing ova-specific IgE (FIG. 5A). This apparent disconnect between the presence of allergen-specific IgE and reactivity to an allergen suggests that other immune changes are behind the suppression of allergic reactions observed here.

To examine these changes, Th2-biased cellular immune responses associated with allergic disease also were evaluated. It was previously reported that allergen-NE immunization suppresses allergen-specific Th2-polarized immunity while inducing Th1 and Th17 immune responses (25-28). To determine if bystander suppression of allergic reactivity was associated with bystander suppression of allergen-specific cellular immunity, mice were sensitized to both ova and peanut, then treated with either ova or peanut in NE. Mesenteric lymph node cells were isolated and stimulated ex vivo with ova to characterize the ova-specific cellular recall response.

Upon stimulation with ova, cells from (ova and peanut)-alum sensitized mice produced significant levels of Th2-type cytokines IL-4 and IL-13, but not IFN-γ (FIG. 5B). Immunotherapy with either ova or peanut in NE reduced ova-specific IL-4 and IL-13 and increased IFN-γ. Because Th2 immunity is required for sustained expression of alarmins and IFN-γ can inhibit alarmin expression, expression of the genes for the alarmins IL-25, IL-33 and TSLP was also evaluated in the small intestine. Immunization with the NE vaccines significantly reduced the expression of I125, I133 and Tslp such that the fold change over expression in naïve mice was approximately 1.

These data indicate that immunization with NE prevents increased alarmin expression normally observed in allergen sensitized mice.

### Example 4

This example demonstrates that intranasal immunization with NE adjuvant suppresses ILC2 populations.

A significant decrease in alarmin expression was observed, including that of IL-33, in animals that received the NE-allergen compositions as compared to sensitized control animals. It has been previously established that IL-33 acts as activation cytokine and regulates ILC2 populations in allergic inflammation (36). ILC2s were quantified in the small intestine lamina propria to determine if reduction in reactivity was associated with reduce ILC2 accumulation in the tissue. ILC2s were increased in the intestine of sensitized mice compared to naïve. Conversely, ILC2s were significantly reduced in NE-ova and NE-PN immunized animals compared to sensitized controls (FIG. 5D). These data suggest that immunization with either NE-ova or NE-PN modulates epithelial IL-33 production, which in turn prevents the accumulation of ILC2s in the tissues.

### Example 5

This example demonstrates that bystander protection induced by NE allergy vaccines requires IFN-γ.

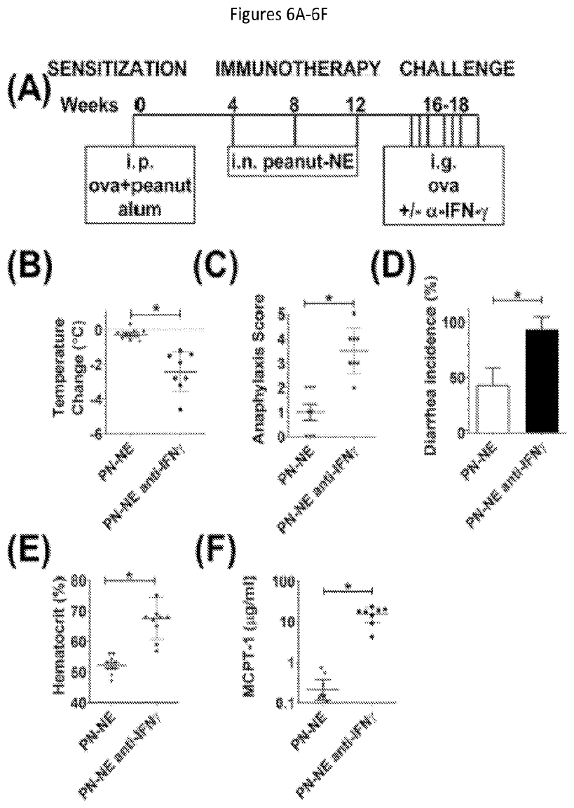

Because NE immunization increased IFN-γ, which has been shown to suppress Th2 immunity and alarmins, it was hypothesized that bystander protection associated with NE was IFN-γ-dependent. Mice were sensitized to ova and peanut and then treated with PN-NE. During the allergen challenge phase in which mice were treated with ova, IFN-γ was depleted. Depletion of IFN-γ during the challenge phase completely abrogated the protection induced by the PN-NE vaccine, as mice treated with anti-IFN-γ antibody had severe reactions to challenge, including decreases in core body temperature and increased clinical symptoms, diarrhea, hematocrit, and MCPT-1 similar to animals not treated with NE (FIG. 6). This demonstrates that NE-induced bystander suppression of allergic disease requires the presence of IFN-γ.

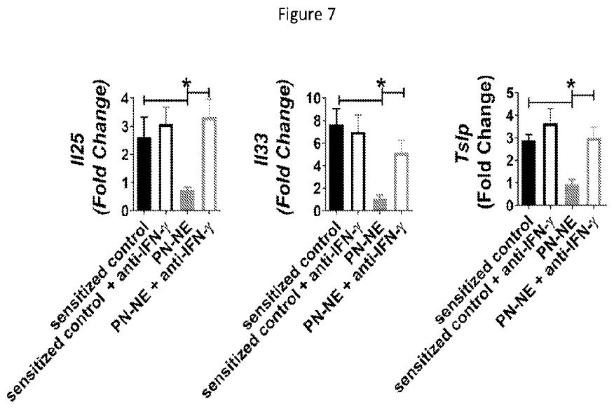

Suppression of alarmin expression by the PN-NE vaccine was also reversed following IFN-γ depletion, as mice that were depleted of IFN-γ had similar expression of alarmins in the small intestine as sensitized mice that did not receive the vaccine (FIG. 7).

The studies described herein demonstrate that NE can be formulated with multiple allergens and lead to allergic suppression of all allergens included in the vaccine. NE was formulated with two allergens and maintained therapeutic efficacy for both foods. Based on the protein loading capacity of NE, a formulation with more than 2 allergens would also be possible and the therapy only has to be administered three to four times to achieve sustained unresponsiveness of at least 4 months (25-27). Given that long-term protection can be achieved with only a few doses administered at monthly intervals, this approach would significantly reduce the burden on patients with multiple food allergies over daily, allergen-specific immunotherapies.

The studies described herein also demonstrate a “bystander” effect in that NE-induced active suppression of allergic immune responses with one allergen also suppressed reactivity to unrelated allergens not included in the vaccine. The bystander protection did require immunization with an allergen to which the animal was previously sensitized, suggesting the non-specific suppression of food allergic reactions required prior sensitization and immune recognition of the vaccine. The mechanism of this effect appeared to be redirection of the underlying immune polarization from a Th2 to a Th1 phenotype, especially since no suppressive activity was demonstrated with treatment using either the NE composition alone or with an immunogen for which there was no pre-existing allergy (hepatitis B antigen). This demonstrates that while the NE-food allergen composition provides bystander protection, it requires the recognition and induction of an antigen-specific immune response.

The NE vaccine compositions described herein suppressed alarmin expression in an IFN-γ-dependent mechanism, and this correlated with suppression of allergic reactivity to bystander allergens. This suggests local NE-induced IFN-γ modulates the small intestine environment to suppress the allergic response. Since these components of the innate immune system function in an antigen-independent manner, this may be responsible for the non-antigen specific bystander effects that confer protection against allergens not included in the NE vaccine composition.

### Example 6

This example demonstrates that NE vaccine containing an individual allergen component from a whole allergen (e.g., peanut) suppresses reactivity to challenge with the whole allergen.

Foods to which patients are allergic often contain multiple different protein allergens (e.g., protein allergens) to which patients can be sensitized. Reactivity to these individual proteins can vary across patients. As detailed herein, immunotherapy with a nanoemulsion (NE) vaccine and one allergen can suppress reactivity and immune responses to other allergens not included in the vaccine. Further experiments were carried out in order to determine if NE vaccine formulated with an individual allergen component from a whole allergen (e.g., peanut) would be able to suppress the reactivity to challenge with the whole allergen. In particular, a NE vaccine formulated with an individual peanut allergen component was generated and tested for its ability to suppress reactivity to whole peanut (e.g., whole peanut extract) that contains multiple additional allergens (e.g., protein allergens).

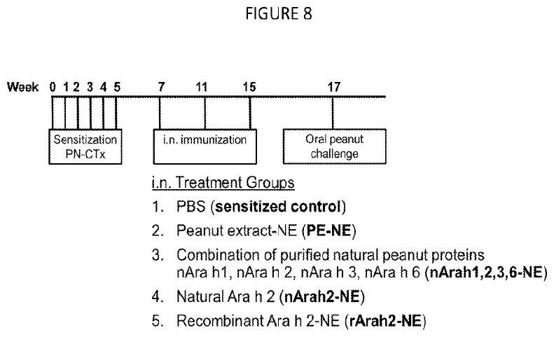

An exemplary experimental design is shown in FIG. 8. C3H/HeJ mice were sensitized to peanut by oral gavage with whole peanut extract and cholera toxin. Mice received 3 intranasal immunizations of one of the following NE vaccines: whole peanut extract in NE; a combination of 4 purified natural peanut proteins (ara h 1, ara h 2, ara h 3 and ara h 6) in NE; or a single major peanut allergen ara h 2 in NE. Both purified natural ara h 2 and recombinant ara h2 were evaluated for their ability to suppress reactivity to peanut.

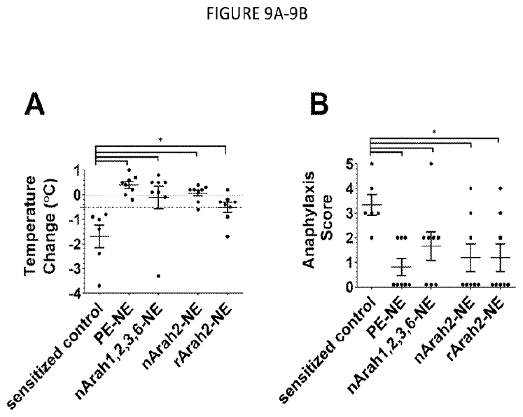

Sensitized mice reacted to oral peanut challenge with a decrease in core body temperature due to hypovolemic shock as well as other clinical symptoms of reactivity, including labored breathing and severe anaphylaxis (FIG. 9). Mice that were immunized with the NE vaccine containing whole peanut extract were significantly protected from reactivity to challenge. Surprisingly, a NE vaccine containing only one individual peanut allergen component (ara h2) was also able to suppresses reactivity to challenge with whole peanut extract. A NE vaccine containing a combination of 4 peanut allergens (natural ara h 1, ara h 2, ara h 3, and ara h 6) was similarly protective. Thus, the disclosure provides that protection from allergic reactivity required immunization with a NE containing only an individual peanut allergen component, and further, that NE vaccines containing either natural ara h 2 or recombinant ara h 2 were similarly protective against reactivity to whole peanut extract.

Accordingly, the disclosure provides that immunization with a vaccine containing a single allergen (e.g., a single major peanut allergen, such as ara h 2) suppresses reactivity to challenge with whole allergen (e.g., a whole extract containing the single allergen plus additional proteins, such as whole peanut extract). No significant differences were observed in efficacy of vaccines containing natural, isolated allergen (e.g., natural ara h 2) versus vaccines containing recombinant allergen (e.g., recombinant ara h 2), indicating that recombinant proteins may be used in place of purified allergens in vaccine formulations.

### REFERENCES

All references, including publications, patent applications, and patents, cited herein are hereby incorporated by reference to the same extent as if each reference were individually and specifically indicated to be incorporated by reference and were set forth in its entirety herein.
1. Gupta R, Holdford D, Bilaver L, Dyer A, Holl J L, Meltzer D. The economic impact of childhood food allergy in the United States. JAMA Pediatr. 2013; 167(11):1026-31.
2. Shamji M H, Durham S R. Mechanisms of immunotherapy to aeroallergens. Clin Exp Allergy. 2011; 41(9):1235-46.
3. Oppenheimer J J, Nelson H S, Bock S A, Christensen F, Leung D Y. Treatment of peanut allergy with rush immunotherapy. J Allergy Clin Immunol. 1992; 90(2):256-62.
4. Nelson H S, Lahr J, Rule R, Bock A, Leung D. Treatment of anaphylactic sensitivity to peanuts by immunotherapy with injections of aqueous peanut extract. J Allergy Clin Immunol. 1997; 99(6 Pt 1):744-51.
5. Investigators PGoC, Vickery B P, Vereda A, Casale T B, Beyer K, du Toit G, et al. AR101 Oral Immunotherapy for Peanut Allergy. N Engl J Med. 2018; 379(21):1991-2001.
6. Jones S M, Sicherer S H, Burks A W, Leung D Y, Lindblad R W, Dawson P, et al. Epicutaneous immunotherapy for the treatment of peanut allergy in children and young adults. J Allergy Clin Immunol. 2017; 139(4):1242-52 e9.
7. Mondoulet L, Dioszeghy V, Vanoirbeek J A, Nemery B, Dupont C, Benhamou P H. Epicutaneous immunotherapy using a new epicutaneous delivery system in mice sensitized to peanuts. Int Arch Allergy Immunol. 2011; 154(4):299-309.
8. Jones S M, Burks A W, Dupont C. State of the art on food allergen immunotherapy: oral, sublingual, and epicutaneous. J Allergy Clin Immunol. 2014; 133(2):318-23.
9. Keet C A, Frischmeyer-Guerrerio P A, Thyagarajan A, Schroeder J T, Hamilton R G, Boden S, et al. The safety and efficacy of sublingual and oral immunotherapy for milk allergy. J Allergy Clin Immunol. 2012; 129(2):448-55, 55 el-5.
10. Burks A W, Jones S M, Wood R A, Fleischer D M, Sicherer S H, Lindblad R W, et al. Oral immunotherapy for treatment of egg allergy in children. N Engl J Med. 2012; 367(3):233-43.
11. Bauer R N, Manohar M, Singh A M, Jay D C, Nadeau K C. The future of biologics: applications for food 393 allergy. J Allergy Clin Immunol. 2015; 135(2):312-23.
12. Galli S J, Tsai M. IgE and mast cells in allergic disease. Nat Med. 2012; 18(5):693-704.
13. Akdis M, Akdis C A. Mechanisms of allergen-specific immunotherapy: multiple suppressor factors at work in immune tolerance to allergens. J Allergy Clin Immunol. 2014; 133(3):621-31.
14. Broide D H. Immunomodulation of Allergic Disease. Annual Review of Medicine. 2009; 60(1):279-91.
15. Casale T, Stokes J. Immunomodulators for allergic respiratory disorders. The Journal of allergy and clinical immunology. 2008; 121:288-96.
16. Bielinska A U, Janczak K W, Landers J J, Makidon P, Sower L E, Peterson J W, et al. Mucosal immunization with a novel nanoemulsion-based recombinant anthrax protective antigen vaccine protects against Bacillus anthracis spore challenge. Infect Immun. 2007; 75(8):4020-9.
17. Makidon P E, Bielinska A U, Nigavekar S S, Janczak K W, Knowlton J, Scott A J, et al. Pre-clinical evaluation of a novel nanoemulsion-based hepatitis B mucosal vaccine. PLoS One. 2008; 3(8):e2954.
18. Passmore C, Makidon P E, O'Konek J J, Zahn J A, Pannu J, Hamouda T, et al. Intranasal immunization with W 5EC adjuvanted recombinant RSV rF-ptn enhances clearance of respiratory syncytial virus in a mouse model. Hum Vaccin Immunother. 2013; 10(3).
19. Lindell D M, Morris S B, White M P, Kallal L E, Lundy P K, Hamouda T, et al. A novel inactivated intranasal respiratory syncytial virus vaccine promotes viral clearance without Th2 associated vaccine-enhanced disease. PLoS One. 2011; 6(7):e21823. 411
20. Hamouda T, Chepurnov A, Mank N, Knowlton J, Chepurnova T, Myc A, et al. Efficacy, immunogenicity and stability of a novel intranasal nanoemulsion-adjuvanted influenza vaccine in a murine model. Hum Vaccin. 2010; 6(7):585-94. 414
21. Bielinska A U, Chepurnov A A, Landers J J, Janczak K W, Chepurnova T S, Luker G D, et al. A novel, killed-virus nasal vaccinia virus vaccine. Clin Vaccine Immunol. 2008; 15(2):348-58.
22. Bielinska A U, Gerber M, Blanco L P, Makidon P E, Janczak K W, Beer M, et al. Induction of Th17 cellular immunity with a novel nanoemulsion adjuvant. Crit Rev Immunol. 2010; 30(2):189-99.
23. Bielinska A U, Makidon P E, Janczak K W, Blanco L P, Swanson B, Smith D M, et al. Distinct pathways of humoral and cellular immunity induced with the mucosal administration of a nanoemulsion adjuvant. J Immunol. 2014; 192(6):2722-33.
24. Makidon P E, Belyakov I M, Blanco L P, Janczak K W, Landers J, Bielinska A U, et al. Nanoemulsion mucosal adjuvant uniquely activates cytokine production by nasal ciliated epithelium and induces dendritic cell trafficking. Eur J Immunol. 2012; 42(8):2073-86.
25. O'Konek J J, Landers J J, Janczak K W, Goel R R, Mondrusov A M, Wong P T, et al. Nanoemulsion adjuvant-driven redirection of TH2 immunity inhibits allergic reactions in murine models of peanut allergy. J Allergy Clin Immunol. 2018; 141(6):2121-31.
26. O'Konek J J, Baker J R, Jr. Treatment of allergic disease with nanoemulsion adjuvant vaccines. Allergy. 2019.
27. O'Konek J J, Landers J J, Janczak K W, Lindsey H K, Mondrusov A M, Totten T D, et al. Intranasal nanoemulsion vaccine confers long-lasting immunomodulation and sustained unresponsiveness in a murine model of milk allergy. Allergy. 2019.
28. Bielinska A U, O'Konek J J, Janczak K W, Baker J R, Jr. Immunomodulation of TH2 biased immunity with mucosal administration of nanoemulsion adjuvant. Vaccine. 2016; 34(34):4017-24.
29. Myc A, Kukowska-Latallo J F, Bielinska A U, Cao P, Myc P P, Janczak K, et al. Development of immune response that protects mice from viral pneumonitis after a single intranasal immunization with influenza A virus and nanoemulsion. Vaccine. 2003; 21(25-26):3801-14.
30. Ahrens R, Osterfeld H, Wu D, Chen C Y, Arumugam M, Groschwitz K, et al. Intestinal mast cell levels control severity of oral antigen-induced anaphylaxis in mice. Am J Pathol. 2012; 180(4):1535-46.
31. Finkelman F D, Katona I M, Mosmann T R, Coffman R L. IFN-gamma regulates the isotypes of Ig secreted during in vivo humoral immune responses. J Immunol. 1988; 140(4):1022-7.
32. Li X M, Serebrisky D, Lee S Y, Huang C K, Bardina L, Schofield B H, et al. A murine model of peanut anaphylaxis: T- and B-cell responses to a major peanut allergen mimic human responses. J Allergy Clin Immunol. 2000; 106(1 Pt 1):150-8. 444
33. Rodriguez B, Prioult G, Hacini-Rachinel F, Moine D, Bruttin A, Ngom-Bru C, et al. Infant gut microbiota is protective against cow's milk allergy in mice despite immature ileal T-cell response. FEMS Microbiol Ecol. 446 2012; 79(1):192-202.
34. Classen D C, Morningstar J M, Shanley J D. Detection of antibody to murine cytomegalovirus by enzyme-linked immunosorbent and indirect immunofluorescence assays. J Clin Microbiol. 1987; 25(4):600-4.
35. Frey A, Di Canzio J, Zurakowski D. A statistically defined endpoint titer determination method for immunoassays. J Immunol Methods. 1998; 221(1-2):35-41.
36. Stier M T, Zhang J, Goleniewska K, Cephus J Y, Rusznak M, Wu L, et al. IL-33 promotes the egress of group 2 innate lymphoid cells from the bone marrow. J Exp Med. 2018; 215(1):263-81.
37. Gupta R S, Springston E E, Warrier M R, Smith B, Kumar R, Pongracic J, et al. The prevalence, severity, and distribution of childhood food allergy in the United States. Pediatrics. 2011; 128(1):e9-17.
38. Gupta R S, Warren C M, Smith B M, Jiang J, Blumenstock J A, Davis M M, et al.

Prevalence and Severity of Food Allergies Among U S Adults. JAMA Netw Open. 2019; 2(1):e185630.
39. Begin P, Winterroth L C, Dominguez T, Wilson S P, Bacal L, Mehrotra A, et al. Safety and feasibility of oral immunotherapy to multiple allergens for food allergy. Allergy Asthma Clin Immunol. 2014; 10(1):1.
40. Andorf S, Manohar M, Dominguez T, Block W, Tupa D, Kshirsagar R A, et al. Feasibility of sustained response through long-term dosing in food allergy immunotherapy. Allergy Asthma Clin Immunol. 2017; 13:52.
41. Wang L F, Chen J S, Hsu C J, Liu C Y, Yu J S, Miaw S C. Antigen-driven bystander effect accelerates epicutaneous sensitization with a new protein allergen. J Biomed Sci. 2009; 16:28.
42. Eisenbarth S C, Zhadkevich A, Ranney P, Herrick C A, Bottomly K. IL-4-dependent Th2 collateral priming to inhaled antigens independent of Toll-like receptor 4 and myeloid differentiation factor 88. J Immunol. 465 2004; 172(7):4527-34.
43. Gregory J A, Shepley-McTaggart A, Umpierrez M, Hurlburt B K, Maleki S J, Sampson H A, et al. Immunotherapy using algal-produced Ara h 1 core domain suppresses peanut allergy in mice. Plant Biotechnol J. 2016; 14(7):1541-50.
44. Storni F, Zeltins A, Balke I, Heath M D, Kramer M F, Skinner M A, et al. Vaccine against peanut allergy based on engineered virus-like particles displaying single major peanut allergens. J Allergy Clin Immunol. 2019.
45. Bannon G A, Cockrell G, Connaughton C, West C M, Helm R, Stanley J S, et al. Engineering, characterization and in vitro efficacy of the major peanut allergens for use in immunotherapy. Int Arch Allergy Immunol. 2001; 124(1-3):70-2.
46. Mondoulet L, Dioszeghy V, Puteaux E, Ligouis M, Dhelft V, Plaquet C, et al. Specific epicutaneous immunotherapy prevents sensitization to new allergens in a murine model. J Allergy Clin Immunol. 2015; 135(6):1546-57 e4.
47. Mondoulet L, Dioszeghy V, Busato F, Plaquet C, Dhelft V, Bethune K, et al. Gata3 hypermethylation and Foxp3 hypomethylation are associated with sustained protection and bystander effect following epicutaneous immunotherapy in peanut-sensitized mice. Allergy. 2019; 74(1):152-64.
48. Dunkin D, Berin M C, Mondoulet L, Tobar S, Yeretssian G, Tordesillas L, et al. Epicutaneous Tolerance Induction to a Bystander Antigen Abrogates Colitis and Ileitis in Mice. Inflamm Bowel Dis. 2017; 23(11):1972-82.
49. Turcanu V, Maleki S J, Lack G. Characterization of lymphocyte responses to peanuts in normal children, peanut-allergic children, and allergic children who acquired tolerance to peanuts. J Clin Invest. 2003; 111(7):1065-484 72.
50. Hammad H, Lambrecht B N. Barrier Epithelial Cells and the Control of Type 2 Immunity. Immunity. 2015; 43(1):29-40.
51. Lee J B, Chen C Y, Liu B, Mugge L, Angkasekwinai P, Facchinetti V, et al. IL-25 and CD4(+) TH2 cells enhance type 2 innate lymphoid cell-derived IL-13 production, which promotes IgE-mediated experimental food 489 allergy. J Allergy Clin Immunol. 2016; 137(4):1216-25 e5.
52. Noval Rivas M, Burton O T, Oettgen H C, Chatila T. IL-4 production by group 2 innate lymphoid cells promotes food allergy by blocking regulatory T-cell function. J Allergy Clin Immunol. 2016; 138(3):801-11 e9.
53. Molofsky A B, Van Gool F, Liang H E, Van Dyken S J, Nussbaum J C, Lee J, et al. Interleukin-33 and Interferon-gamma Counter-Regulate Group 2 Innate Lymphoid Cell Activation during Immune Perturbation. Immunity. 2015; 43(1):161-74.
54. Li BWS, de Bruijn MJW, Lukkes M, van Nimwegen M, Bergen I M, KleinJan A, et al. T cells and ILC2s are major effector cells in influenza-induced exacerbation of allergic airway inflammation in mice. Eur J Immunol. 2019; 49(1):144-56.
55. Starkey M R, McKenzie A N, Belz G T, Hansbro P M. Pulmonary group 2 innate lymphoid cells: surprises and challenges. Mucosal Immunol. 2019; 12(2):299-311.

The use of the terms “a” and “an” and “the” and “at least one” and similar referents in the context of describing the aspects of the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of embodiments of the disclosure.

Preferred embodiments of this disclosure are described herein, including the best mode known for carrying out aspects of the disclosure. Variations of those preferred embodiments may become apparent to those of ordinary skill in the art upon reading the foregoing description. It is expected that skilled artisans will employ such variations as appropriate, and therefore it is intended that the disclosure be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of inhibiting an allergic reaction to two or more food allergens in a subject, which method comprises administering to the subject a composition comprising a nanoemulsion and at least one of the two or more food allergens.

2. The method of claim 1, wherein the composition comprises one of the two or more food allergens.

3. The method of claim 1, wherein the composition comprises all of the two or more food allergens.

4. The method of claim 1, wherein the two or more food allergens are selected from milk, eggs, fish, crustacean shellfish, tree nuts, peanuts, wheat, and soya.

5. The method of claim 1, wherein the nanoemulsion comprises:
(a) a poloxamer surfactant or polysorbate surfactant;
(b) an organic solvent;
(c) a halogen-containing compound;
(d) oil, and
(e) water.

6. The method of claim 5, wherein the nanoemulsion comprises:
a) about 3 vol. % to about 15 vol. % of a poloxamer surfactant or polysorbate surfactant;
b) about 3 vol. % to about 15 vol. % of an organic solvent;
c) about 0.5 vol. % to about 1 vol. % of a halogen-containing compound;
d) about 3 vol. % to about 90 vol. % of an oil; and
e) about 5 vol. % to about 60 vol. % of water.

7. The method of claim 1, wherein the subject is a human.

8. The method of claim 1, wherein the composition is administered to the subject intranasally.

9. The method of claim 1, wherein the composition is administered two or more times to the subject.

10. The method of claim 1, wherein the expression of Th2 type cytokines is reduced in the subject.

11. The method of claim 1, wherein the expression of alarmins is reduced in the subject.

12. The method of claim 1, wherein the expression of IFN-γ is induced in the subject.

13. A method of inhibiting an allergic reaction to a whole food allergen comprising a plurality of individual allergen components, which method comprises administering to the subject a composition comprising a nanoemulsion and at least one of the plurality of individual allergen components.

14. The method of claim 13, wherein the composition comprises a single allergen of the plurality of individual allergen components.

15. The method of claim 13, wherein the composition comprises two different allergens, three different allergens, or four different allergens of the plurality of individual allergen components.

16. The method of claim 13, wherein the whole food allergen is selected from milk, egg, fish, crustacean shellfish, tree nut, peanut, wheat, and soya.

17. The method of claim 13, wherein the whole food allergen is peanut.

18. The method of claim 17, wherein the composition comprises a nanoemulsion and a single peanut allergen selected from ara h 1, ara h 2, ara h 3, and ara h 6.

19. The method of claim 18, wherein the composition comprises a nanoemulsion and ara h 2.

20. The method of claim 13, wherein at least one of the plurality of individual allergen components is a purified allergen or a recombinant allergen.

21. The method of claim 13, wherein the nanoemulsion comprises:
(a) a poloxamer surfactant or polysorbate surfactant;
(b) an organic solvent;
(c) a halogen-containing compound;
(d) oil, and
(e) water.

22. The method of claim 21, wherein the nanoemulsion comprises:
a) about 3 vol. % to about 15 vol. % of a poloxamer surfactant or polysorbate surfactant;
b) about 3 vol. % to about 15 vol. % of an organic solvent;
c) about 0.5 vol. % to about 1 vol. % of a halogen-containing compound;
d) about 3 vol. % to about 90 vol. % of an oil; and
e) about 5 vol. % to about 60 vol. % of water.

23. The method of claim 13, wherein the subject is a human.

24. The method of claim 13, wherein the composition is administered to the subject intranasally.

25. The method of claim 13, wherein the composition is administered two or more times to the subject.

26. The method of claim 13, wherein the expression of Th2 type cytokines is reduced in the subject.

27. The method of claim 13, wherein the expression of alarmins is reduced in the subject.

28. The method of claim 13, wherein the expression of IFN-γ is induced in the subject.
